(19) United States
(12) Patent Application Publication
(10) Pub. No.: US 2026/0002196 A1
(43) Pub. Date: Jan. 1, 2026

(54) Programmable Cleavage of RNA

(71) Applicant: New England Biolabs, Inc., Ipswich, MA (US)

(72) Inventors: Sebastian Grünberg, Salem, MA (US); Eric Hunt, Danvers, MA (US); Lizhi Liu, Beverly, MA (US); Erbay Yigit, Boxford, MA (US); Ivan R. Correa, JR., Hamilton, MA (US)

(73) Assignee: New England Biolabs, Inc., Ipswich, MA (US)

(21) Appl. No.: 18/969,910

(22) Filed: Dec. 5, 2024

Related U.S. Application Data
(60) Provisional application No. 63/666,492, filed on Jul. 1, 2024.

(51) Int. Cl. C12Q 1/6806

(52) U.S. Cl. CPC C12Q 1/6806

(57) ABSTRACT

The present disclosure relates, according to some embodiments, to compositions. methods, and kits for specifically cleaving target polynucleotides (e.g., RNA) into fragments short enough for analysis. for example, by LC-MS/MS.

### CROSS-REFERENCE TO RELATED APPLICATIONS

This application also claims priority to U.S. Provisional Application No. 63/666,492 filed Jul. 1, 2024, the contents of which are hereby incorporated in their entirety by reference.

### SEQUENCE LISTING STATEMENT

This disclosure includes a Sequence Listing submitted electronically in .xml format under the file name “NEB-483.xml” created on Nov. 26, 2024, and having a size of 60,454 bytes. This Sequence Listing is incorporated herein in its entirety by this reference.

### BACKGROUND

Argonaute proteins have an ability to bind small single-stranded 5′-phosphorylated nucleic acids which provide base-pairing specificity for targeting complementary single-stranded targets. Despite the fact that prokaryotes lack RNA interference pathways, many bacterial and archaeal organisms also possess Argonaute proteins implying a different biological role and/or mechanism of action for these proteins within a cell. Multiple recent studies suggest that prokaryotic Argonautes in vivo function as defense systems against foreign genetic elements. Prokaryotic Agos (pAgos) represent a very diverse group of proteins and based on the presence or absence of the basic domains can be divided into two major groups—the short pAgos and the long pAgos. All known active pAgos belong to a long Ago group and, similar to eAgos, consist of four essential domains, N-terminal, PAZ, MID and PIWI. In contrast to eAgos, which use RNA guides to exclusively target RNA, different bacterial Agos have been shown to bind either RNA or DNA guides and to cleave either RNA or DNA targets, whereas some archaeal Agos exclusively utilize DNA guides for cleavage of DNA targets.

In addition to a guided cleavage of complementary targets, many pAgos exhibit non-specific nuclease activity when they are not associated with the guides. TtAgo co-purifies with DNA sequences that are preferentially derived from its own expression plasmid, but only if the Argonaute is catalytically active. Based on these and similar studies, the non-specific activity of pAgos was implicated in cellular function required for guide processing. While the physiological mechanism for DNA guide processing in vivo still remains ambiguous, the most recent study of Argonaute from the mesophilic bacterium Clostridium butyricum shows that its nucleolytic activity cooperates with cellular double-strand break repair machinery in generation of small DNAs that later can be used as guides by this Argonaute.

### SUMMARY

The present disclosure relates to systems, apparatus, compositions, and/or methods cleaving a single-stranded target RNA. For example, a method may include contacting a single-stranded target RNA comprising a guide-recognition sequence; an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and a guide having a sequence complementary to the guide-recognition sequence, wherein the guide is operatively bound to the Argonaute forming an Argonaute:guide complex, to produce cleavage products (e.g., comprising one or more fragments of the single-stranded RNA). A targeted single-stranded RNA may have any desired length and any desired number of guide recognition sequences. For example, a single-stranded RNA may comprise at least 5000 nucleotides, wherein the single-stranded RNA has one guide recognition sequence. A single-stranded RNA, in some embodiments, may comprise one or more modified nucleotides. According to some embodiments, a guide sequence may be selected from any of SEQ ID NOS: 2-11, 13-22, 24-28, and 30-37.

In some embodiments, a single-stranded RNA may comprise a messenger RNA (mRNA), a ribosomal RNA (rRNA), a transfer RNA (tRNA), a small RNA (sRNA), a microRNA (miRNA), a long noncoding RNA (IncRNA), short noncoding RNA (snoRNA, snRNA miRNA, piRNA, siRNA), a circular RNA (circRNA), a transfer RNA (tRNA), an aptamer RNA, an antisense RNA, a silencing RNA (siRNA), a guide RNA (gRNA), or a therapeutic RNA. A single-stranded RNA, in some embodiments, may be or comprise a polycistronic template comprising at least five guide recognition sequences that each corresponds to the Argonaute: guide complex. A single-stranded RNA may comprise one or more modified nucleotides. Cleavage products may include, according to some embodiments, an IVT template (e.g., where the template for transcription is an RNA template) or an IVT product. In some embodiments, one or more cleavage products may be analyzed (e.g., by electrophoretic, photometric, and/or mass spectrometric analysis).

The present disclosure further relates to compositions for cleaving a target (e.g., a target RNA). For example, a composition may comprise a single-stranded target RNA comprising a guide-recognition sequence; an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and a guide having a sequence complementary to a single-stranded target RNA guide-recognition sequence, wherein the guide is operatively bound to the Argonaute forming an Argonaute: guide complex. A composition may further comprise a buffer, for example, a non-naturally occurring buffer (e.g., HEPES, MES, MOPS, TAPS, tricine, Tris, ACES, ADA, BES, Bicine, CAPS, CHES, DIPSO, EPPS, MOPSO, PIPES, POPSO, TAPS, and TAPSO). A composition may have any desired form. For example, a composition may have a form selected from a dried form, a freeze dried form, a lyophilized form, a crystalline form, an aqueous form, and an immobilized form.

The present disclosure further relates to kits for cleaving a target (e.g., a target RNA). For example, a kit may comprise a single-stranded target RNA comprising a guide-recognition sequence; an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and a guide having a sequence complementary to at least a portion of a single-stranded target RNA, wherein the guide has a sequence selected from any of SEQ ID NOS: 2-11, 13-22, 24-28, and 30-37. A kit, according to some embodiments, may further comprising a buffering agent selected from HEPES, MES, MOPS, TAPS, tricine, Tris, ACES, ADA, BES, Bicine, CAPS, CHES, DIPSO, EPPS, MOPSO, PIPES, POPSO, TAPS, and TAPSO. In some embodiments, an Argonaute and/or a guide included in a kit may have any desired form (e.g., a dried form, a freeze-dried form, a lyophilized form, a crystalline form, an aqueous form, and an immobilized form).

### BRIEF DESCRIPTION OF THE FIGURES

FIG. 1A illustrates a target RNA substrate (top row) and products of 8 example reactions (rows 2-9), each of which combined this substrate, an Ago, and one of 8 unique guides (G1-G8). As shown, each reaction (R1-R8) results in the cleavage of the target RNA into two distinct cleavage fragments whose lengths are determined by the guide-programmed cleavage site.

FIG. 1B shows example results of cleaving a 1678 nt mRNA molecule (SEQ ID NO: 1) with Mucilaginibacter paludis (Mpa) Ago. Each reaction was performed with a unique guide with each guide selected to create a cleavage site approximately 100 nucleotides from the flanking cleavage site(s) as illustrated in FIG. 1A. Reaction products were size fractionated on a gel in which lane 1 contains a sizing ladder, lane 2 contains products of a reaction that included MpaAgo, but excluded a guide, and lanes 3-12 contain products of reactions that included one of guides 90.1-90.10 (SEQ ID NO:2 to SEQ ID NO:11), respectively, and MpaAgo. Bands representing cleaved RNA products or guide RNAs are marked. The cleavage efficiency in percent is shown above the gel.

FIG. 1C shows results of cleaving a 1678 nt mRNA molecule (SEQ ID NO:1) with Clostridum perfringens (Cpe) Ago. Each reaction was performed with a unique guide with each guide selected to create a cleavage site approximately 100 nucleotides from the flanking cleavage site(s) as illustrated in FIG. 1A. Reaction products were size fractionated on a gel in which lane 1 contains a sizing ladder, lane 2 contains products of a reaction that included CpeAgo, but excluded a guide, and lanes 3-12 contain products of reactions that included one of guides 90.1-90.10 (SEQ ID NOS: 2-11), respectively, and CpeAgo. Bands containing cleaved RNA products or guide RNAs are marked. The cleavage efficiency in percent is shown above the gel.

FIG. 2A shows the results of a series of example reactions in which FLuc mRNA (SEQ ID NO: 12) was contacted with MpaAgo and one of ten guides (F1.1-F1.10; SEQ ID NOS: 13-22), the sequences of which were selected to be complementary to regions of the FLuc mRNA that were 72 nt apart from each other. Reaction products were size fractionated on a gel in which lane 1 contains a sizing ladder, lane 2 contains products of a reaction that included MpaAgo, but excluded a guide, and lanes 3-12 contain products of reactions that included one of guides F1.1-F1.10, respectively, and MpaAgo. Bands containing cleaved RNA products or guide RNAs are marked. The cleavage efficiency in percent is shown above the gel.

FIG. 2B shows the results of a series of example reactions in which a pSG95 epo mRNA (SEQ ID NO:23) was contacted with MpaAgo and one of five guides (95.1-95.10; SEQ ID NOS: 24-28), the sequences of which were selected to be complementary to regions of the pSG95 epo mRNA that were 100 nt apart from each other. Reaction products were size fractionated on a gel in which lane 1 contains a sizing ladder, lane 2 contains products of a reaction that included MpaAgo, but excluded a guide, and lanes 3-7 contain products of reactions that included one of guides 95.1-95.5, respectively, and MpaAgo. Bands containing cleaved RNA products or guide RNAs are marked. The cleavage efficiency in percent is shown above the gel.

FIG. 3A and FIG. 3B show the results of a series of example reactions in which pSG90 saRNA was contacted with MpaAgo (FIG. 3A) or CpeAgo (FIG. 3B) and one of nine guides (1-9), the sequences of which were selected to be unique relative to one another and non-complementary to any portion of the pSG90 saRNA. Reaction products were size fractionated on a gel. The uncut pSG90 substrate is marked with “*” and the guides are marked with “**”.

FIGS. 4A, 4B, and 4C show examples of multiplexing embodiments in which a single reaction includes a substrate RNA, two or more guides, and a single selected Ago where approximately equal fractions of the population of Ago molecules are loaded with each of the guides present.

FIG. 4A illustrates a hypothetical multiplex reaction in which guides programmed to target distinct sequences were mixed with an Ago and a mRNA or saRNA substrate in a single reaction. As shown, the guides are designed to specifically bind sites on the substrate that are 70-110 nts away from each other and from the 5′ and 3′ ends such that the single reaction would yield a pool of a defined number of different fragments of the substrate, all of which are about 70-110 nts in size. The identity of the fragments may be determined by LC-MS/MS sequence analysis.

FIG. 4B shows gel fractionated products of a series of example reactions in which pSG90 saRNA was contacted with MpaAgo alone (lane 1), MpaAgo and guide 90.1 (lane 2), MpaAgo and guide 90.2 (lane 3), or MpaAgo and equimolar amounts of guides 90.1 and 90.2 (lane 4). The unmarked lane on the left contains a sizing ladder. Bands containing uncut pSG90 saRNA substrate and guide RNAs are marked. Bands representing the size of the programmed cleavage fragments are marked with “*”.

FIG. 4C shows gel fractionated products of example reactions in which FLuc mRNA was contacted with MpaAgo and a pool of 24 guides (F1.1-F1.24) without (lane 2) or with (lane 3) the addition of glycerol and other crowding agents. The reaction in lane 1 contains MpaAgo but no guides. Cleaved RNA fragments are labeled to the right of the gel: FLuc mRNA=full length, uncleaved FLuc mRNA, 144 nt=two 72 nt fragments that did not get fully cleaved, 81 nt=fragment of the 3′ terminus of the FLuc mRNA, 72 nt=programmed fragment size for all but the 5′ and 3′ termini of the RNA, 29 nt=fragment of the 5′ terminus of the FLuc mRNA, guides (16 nt)=guides).

FIG. 4D shows the percentage of sequence coverage that was achieved in LC-MS/MS for an example reaction containing the 24 multiplexed guides (left bar) and the 24 multiplexed guides and a mix of crowding agents.

FIGS. 5A and 5B show the gel fractionated products of a series of example reactions in which pSG90 saRNA was contacted with MpaAgo and guide 90.9 for the indicated times in minutes at 4° C. or 24° C. (FIG. 5A) or 37° C. or 50° C. (FIG. 5B) with uncut substrate pSG90 saRNA, cleavage products, and guides marked.

FIG. 6 shows the gel fractionated products of a series of example reactions in which pSG90 saRNA substrate was contacted with MpaAgo and guide 90.1 at 50° C. followed by DNase digestion. The leftmost lane contains a sizing ladder. Lane 1 contains the products of a reaction that omitted a DNase, lane 2 the products of a reaction that included E. coli exonuclease I, lane 3, the products of a reaction that included Lambda exonuclease, and lane 4, the products of a reaction that included both E. coli exonuclease I and Lambda exonuclease. Cleavage products and guides are marked.

FIG. 7A illustrates a hypothetical reaction in which an Ago comprising a bound sequence specific guide binds and cleaves a 33-nt RNA substrate to form a first cleavage product comprising the 5′ portion of the substrate, namely 20 nucleotides, and a second product comprising the 3′ portion of the substrate, namely the remaining 13 nts. FIG. 7B shows the results of a series of example reactions in which a 33 nt capped synFLuc RNA was contacted with MpaAgo or CpeAgo and one of four guides.

FIG. 7C shows a workflow in which an mRNA is contacted by an Ago that is programmed to cleave downstream of the 5′ end of the mRNA. After successful cleavage, the fragment containing the 5′ end is purified and analyzed by LC-MS/MS.

FIG. 7D (left panel) shows the gel fractionated products of a reaction as described in FIG. 7C, in which MpaAgo was programmed to cleave 29 nts downstream of the 5′ end of an FLuc mRNA. The reaction in the lane marked “−” contained FLuc mRNA that had not been enzymatically capped with FCE prior to being contacted with MpaAgo. The reaction in the lane marked “+” contained FLuc mRNA that had been capped with FCE prior to being contacted by MpaAgo. FIG. 7D (right panel) shows a heatmap of the LC-MS/MS analysis of the 5′ fragments, in which the lane captioned “FCE” shows that in this example reaction with FCE, over 99% of the mRNA was capped with a m7Gppp cap, while the no enzyme control (lane marked “NE”) contained over 94% of triphosphate 5′ ends.

FIG. 7E (left panel) shows the gel fractionated products of a reaction as described in FIG. 7C, in which MpaAgo was programmed to cleave 30 nts downstream of the 5′ end of an pSG90 mRNA. The reaction in the lane marked “−” contained Ago and uncapped pSG90 mRNA, and the reaction in the lane marked “+” contained Ago and FCE-capped pSG90 mRNA. FIG. 7E (right panel) shows a heatmap of the LC-MS/MS analysis of the 5′ fragments. mRNA that was enzymatically capped with FCE showed approximately 88% capping efficiency with m7Gppp, with the balance 7% diphosphate, 2% Gppp, and 3% of uncapped triphosphate capping intermediates. mRNAs in the “no enzyme” control reaction contained approximately 95% uncapped triphosphate and 4% diphosphate 5′ ends.

FIG. 8A illustrates a hypothetical reaction in which an Ago programmed with a guide to having a sequence complementary to a 3′ portion of an RNA (for example a guide target sequence embedded in the poly(A) tail.

FIG. 8B illustrates a hypothetical reaction in which an Ago programmed with a guide to having a sequence complementary to a portion of an RNA upstream of the poly(A) tail. In both FIGS. 8A and 8B, the programmed Ago binds to and cleaves the substrate RNA to produce reaction products having uniform 3′ ends. Cleaved 3′ fragments may be removed and their composition/sequence analyzed by LC-MS/MS.

FIG. 8C shows a section of an example poly(A) tail sequence (nucleotides 602-671 of SEQ ID NO:37) and a selection of example guides (D834 (SEQ ID NO:59) having a target upstream of the poly(A) tail and D758 (SEQ ID NO:48), D759 (SEQ ID NO:49), D760 (SEQ ID NO:50), and D761 (SEQ ID NO:51) having targets within the poly(A) tail. The putative cleavage sites of a pAgo bound to each guide is shown above the sequence.

FIG. 8D shows the gel fractionated products of a series of example reactions in which pSG95 mRNA substrate (SEQ ID NO:37) was contacted with MpaAgo having guides complementary to various positions in the target sequence (SEQ ID NOS: 48-51; marked “D758-D761”) or with MpaAgo without a guide (marked “−”).

FIG. 8E shows a section of an example poly(A) tail sequence (nucleotides 602-663 of SEQ ID NO:60) with two guides (D758 and D834) positioned according to their complementarity. The respective expected cleavage sites and the length of the putative cleavage fragments are indicated above the sequence.

FIG. 8F shows the identified 3′ cleavage fragments after an example cleavage reaction with an MpaAgo programmed with the D758 (left panel) or the D834 (right panel) guides. Black peaks represent poly(A) tail containing sequences, grey peaks indicate unassigned fragments. The length in nucleotides of the respective fragments is indicated on top of each peak. Peaks labeled with an asterisk resemble the expected, template encoded length of the poly(A) tail.

FIG. 9A shows an example method of cleaving a polycistronic RNA comprising contacting an Ago, a guide, and an RNA comprising copies (here, 5) of RNA module 1, each module flanked by at least one sequence complementary to the guide to form cleavage products, wherein the cleavage products comprise separated copies (here, 5) of RNA module 1 and may further include separate 5′ and 3′ end fragments as shown.

FIG. 9B shows an example method of cleaving a polycistronic RNA comprising contacting an Ago, a guide, and an RNA comprising different RNA modules (here, 1-5), each module flanked by at least one sequence complementary to the guide to form cleavage products, wherein the cleavage products comprise separated RNA modules (here, 1, 2, 3, 4, and 5) and may further include separate 5′ and 3′ end fragments as shown. In some embodiments, a method may include two or more guides and module flanking sequences may be varied to correspond to one or more of the included guides. For example, module 1 may be flanked by sequences complementary to guide 1 and a portion of the sequence 3′ of module 2 may correspond to guide 2 and (optionally) a portion of the sequence 3′ of module 3 may correspond to guide 3 and so on.

FIG. 9C is a schematic image of the pSG106 mRNA (IVT; SEQ ID NO:52), in which target sites for MpaAgo loaded with guide 770 (SEQ ID NO: 53) are indicated by asterisks, and the fragments to be released from a cleavage reaction are shown.

Expected/programmed fragment sizes are as follows: frag 1, 99 nts; frag 2, 201 nts; frag 3, 301 nts; frag 4, 405 nts; frag 5, 514 nts; and frag 6, 56 nts.

FIG. 9D shows the gel fractionated products of example reactions containing MpaAgo without guide 770 (lane 1), with guide 770 (lane 2), and with guide 770 supplemented with a crowding agent (lane 3). Predicted cleavage fragments are labeled on the right (frag 1-6).

FIG. 10 shows an example screening method for identification and/or purification of an RNA of interest that is included in an RNA pool. As illustrated, a method may comprise contacting the RNA pool with a 3′ hydroxyl blocking reagent to form a 3′ blocked RNA pool, contacting the 3′ blocked RNA pool with an Ago and guide, the guide having a sequence complementary to at least a portion (e.g., a unique portion) of the RNA of interest to form a cleaved RNA of interest comprising a 3′-OH, contacting the cleaved RNA of interest with a 3′-OH labelling agent (here, biotin-ATP and poly(A) polymerase) to form a labeled RNA of interest, and enriching (here, with streptavidin) and/or detecting the labeled RNA of interest.

FIG. 11A shows an example adapter dimer (R594; SEQ ID NO:55) that can form (e.g., by ligation of the 5′ adapter directly to the 3′ adapter with no insert between) during RNA library preparation for Illumina sequencing, with the 5′ RNA part having the sequence of the NEBNext 5′ SR adaptor and the 3′ DNA part having the sequence of the NEBNext 3′ SR adaptor. For visualization purposes, this sequence comprises a fluorescent label on its 5′ end.

FIG. 11B shows example DNA guides D826 (SEQ ID NO:56), D827 (SEQ ID NO: 57), and D828 (SEQ ID NO:58), the respective cleavage sites in the R594 substrate (SEQ ID NO: 55) when used to program MpaAgo (dotted lines), and the lengths of the respective cleavage products (solid lines with arrows above the sequence).

FIG. 11C shows the gel fractionated products of example reactions containing R594 substrate (SEQ ID NO:55), MpaAgo (except for the reaction shown in lane 8, which did not contain Ago), and D826/D827/D828 (SEQ ID NOS: 56-58) guides either at a 1:1 ratio or 1:5 ratio of MpaAgo to guide as indicated above the gel. The lengths of the uncleaved substrate (48 nt) and the cleavage products are indicated at the right of the gel.

### BRIEF DESCRIPTION OF THE SEQUENCES

Some embodiments of this disclosure relate to the following provided sequences of example polynucleotides and/or example polypeptides.

SEQ ID NO: 1 is an example pSG90 saRNA.

SEQ ID NO: 2 is an example guide, namely DNA guide 90.1.

SEQ ID NO: 3 is an example guide, namely DNA guide 90.2.

SEQ ID NO: 4 is an example guide, namely DNA guide 90.3.

SEQ ID NO: 5 is an example guide, namely DNA guide 90.4.

SEQ ID NO: 6 is an example guide, namely DNA guide 90.5.

SEQ ID NO: 7 is an example guide, namely DNA guide 90.6.

SEQ ID NO: 8 is an example guide, namely DNA guide 90.7.

SEQ ID NO: 9 is an example guide, namely DNA guide 90.8.

SEQ ID NO: 10 is an example guide, namely DNA guide 90.9.

SEQ ID NO: 11 is an example guide, namely DNA guide 90.10.

SEQ ID NO: 12 is an example FLuc mRNA.

SEQ ID NO: 13 is an example guide, namely DNA guide F1.1.

SEQ ID NO: 14 is an example guide, namely DNA guide F1.2.

SEQ ID NO: 15 is an example guide, namely DNA guide F1.3.

SEQ ID NO: 16 is an example guide, namely DNA guide F1.4.

SEQ ID NO: 17 is an example guide, namely DNA guide F1.5.

SEQ ID NO: 18 is an example guide, namely DNA guide F1.6.

SEQ ID NO: 19 is an example guide, namely DNA guide F1.7.

SEQ ID NO: 20 is an example guide, namely DNA guide F1.8.

SEQ ID NO: 21 is an example guide, namely DNA guide F1.9.

SEQ ID NO: 22 is an example guide, namely DNA guide F1.10.

SEQ ID NO: 23 is an example guide, namely DNA guide F1.11.

SEQ ID NO: 24 is an example guide, namely DNA guide F1. 12.

SEQ ID NO: 25 is an example guide, namely DNA guide F1.13.

SEQ ID NO: 26 is an example guide, namely DNA guide F1.14.

SEQ ID NO: 27 is an example guide, namely DNA guide F1.15.

SEQ ID NO: 28 is an example guide, namely DNA guide F1.16.

SEQ ID NO: 29 is an example guide, namely DNA guide F1.17.

SEQ ID NO: 30 is an example guide, namely DNA guide F1.18.

SEQ ID NO: 31 is an example guide, namely DNA guide F1.19.

SEQ ID NO: 32 is an example guide, namely DNA guide F1.20.

SEQ ID NO: 33 is an example guide, namely DNA guide F1.21.

SEQ ID NO: 34 is an example guide, namely DNA guide F1.22.

SEQ ID NO: 35 is an example guide, namely DNA guide F1.23.

SEQ ID NO: 36 is an example guide, namely DNA guide F1.24.

SEQ ID NO: 37 is an example pSG95 epo mRNA.

SEQ ID NO: 38 is an example guide, namely DNA guide 95.1.

SEQ ID NO: 39 is an example guide, namely DNA guide 95.2.

SEQ ID NO: 40 is an example guide, namely DNA guide 95.3.

SEQ ID NO: 41 is an example guide, namely DNA guide 95.4.

SEQ ID NO: 42 is an example guide, namely DNA guide 95.5.

SEQ ID NO: 43 is an example synfluc 8.1 substrate.

SEQ ID NO: 44 is an example guide, namely DNA guide 8.1L15-A.

SEQ ID NO: 45 is an example guide, namely DNA guide 8.1L15-T.

SEQ ID NO: 46 is an example guide, namely DNA guide 8.1L15-G.

SEQ ID NO: 47 is an example guide, namely DNA guide 8.1L15-C.

SEQ ID NO: 48 is an example guide, namely DNA guide 758.

SEQ ID NO: 49 is an example guide, namely DNA guide 759.

SEQ ID NO: 50 is an example guide, namely DNA guide 760.

SEQ ID NO: 51 is an example guide, namely DNA guide 761.

SEQ ID NO: 52 is an example pSG106 mRNA.

SEQ ID NO: 53 is an example guide, namely DNA guide 770.

SEQ ID NO: 54 is an example guide, namely DNA guide 831.

SEQ ID NO: 55 is an example RNA/DNA adapter dimer, namely R594.

SEQ ID NO: 56 is an example guide, namely D826.

SEQ ID NO: 57 is an example guide, namely D827.

SEQ ID NO: 58 is an example guide, namely D828.

SEQ ID NO: 59 is an example guide, namely D834.

SEQ ID NO: 60 is an example pSG120 mRNA.

### DETAILED DESCRIPTION

Fueled by the recent success of mRNA vaccines against COVID-19, the field of RNA-based therapeutics is growing exponentially and considered the future of modern medicine. Alongside the conventional mRNA vaccines/therapeutics, self-amplifying RNA (saRNA) as another type of RNA-based therapeutics is quickly gaining popularity. saRNAs are larger molecules (10-25 kb) than conventional mRNA therapeutics as they encode the replicase machinery in addition to the therapeutic protein/vaccine antigen that amplifies synthetic transcripts in situ. saRNA are deemed the next generation of RNA therapeutics, as they have multiple advantages over conventional mRNAs (low dosage, reduced side effects, long lasting outcomes).

However, due to their large size, characterizing or performing any form of conclusive quality control on saRNA using mass spectrometry poses a significant challenge. Current methods to analyze conventional mRNAs (digestion with nucleotide-specific RNases like T1 followed by intact mass analysis by LC-MS/MS) cannot be applied to long saRNA, because the resulting pool of short RNA oligomers (15-20 bases) is too complex to deconvolute efficiently and/or effectively.

The present disclosure relates, in some embodiments, to compositions, methods, systems, and kits for cleaving long RNA molecules into fragments that are a) short enough to be analyzed by LC-MS/MS, and b) long enough to reduce the complexity of the data by using programable ribonucleases. These enzymes may be used to either completely fragment long RNAs for a comprehensive sequence analysis of the substrate/therapeutic, or to “fingerprint” RNA by generating substrate-specific fragments that allow for easy identification or RNA species. In addition, programable nucleases may be used to assess the efficiency of 5′ capping and the homogenization of the 3′ ends of regular mRNA molecules.

The present disclosure relates, in some embodiments, to compositions, methods, systems, and kits that include programmable endoribonucleases (e.g., argonautes with single-stranded DNA guides). In some embodiments, cleavage may occur at a physiological temperature (e.g., at temperatures from 4° C. to 50° C.) optionally, in the absence or presence of other proteins (e.g., in the absence or presence of a helicase, a DNA-binding protein, a single-stranded RNA-binding protein) and/or in the absence or presence of chemical agents. Examples of chemical agents include alkali, dimethylsulfoxide (DMSO), urea, and formamide. Conditions may otherwise be used or adjusted to permit or favor cleavage of and/or to destabilize double stranded regions of a substrate RNA, for example, by contacting the substrate RNA with media having a low or high ionic strength, with air, and/or with glass.

The present disclosure relates, in some embodiments, compositions comprising an Argonaute, a guide, and a substrate RNA (e.g., a single-stranded RNA). Where a substrate sequence is known, guides may be chosen (e.g., designed, synthesized, selected) to cleave the substrate at a specific location or chosen to avoid cleavage at a specific location (or avoid cleavage at any location) in the substrate sequence. A composition may include or exclude other proteins (e.g., single-stranded polynucleotide binding proteins, helicases, polymerases, capping enzymes, methylases, and/or other enzymes). For example, a composition may exclude a single-stranded binding protein. A composition may include or exclude other components (e.g., genomic DNA, plasmid DNA, cellular DNA, buffers, nucleotides, salts).

### General Considerations

Aspects of the present disclosure can be understood in light of the provided descriptions, figures, sequences, embodiments, section headings, and examples, none of which should be construed as limiting the entire scope of the present disclosure in any way. Accordingly, the innovations set forth herein should be construed in view of the full breadth and spirit of the disclosure.

Each of the individual embodiments described and illustrated herein has discrete components and features which can be readily separated from or combined with the components and/or features of any of the other several embodiments without departing from the scope or spirit of the present teachings. Lists of example species within a particular genus may vary in length at different places throughout the disclosure. Species lists shortened for convenience shall not be construed to exclude example species listed elsewhere in the specification. Any recited method can be carried out in the order of events recited or in any other order which is logically possible.

Unless otherwise expressly stated to be required herein, each component, feature, and method step disclosed herein is optional and the disclosure contemplates embodiments in which each optional element may be expressly excluded. As such, this statement is intended to serve as antecedent basis for use of such exclusive terminology as “solely,” “only” and the like in connection with the recitation of claim elements or use of a “negative” limitation. It is further intended to serve as antecedent basis for use of such elective terminology as “optionally” and the like in connection with the recitation of one or more claim elements.

Unless otherwise defined, all technical and scientific terms used herein have the same meaning as commonly understood by one of ordinary skill in the art to which this disclosure belongs. Still, certain terms are defined herein with respect to embodiments of the disclosure and for the sake of clarity and ease of reference.

Sources of commonly understood terms and symbols may include: standard treatises and texts such as Kornberg and Baker, DNA Replication, Second Edition (W. H. Freeman, New York, 1992); Lehninger, Biochemistry, Second Edition (Worth Publishers, New York, 1975); Strachan and Read, Human Molecular Genetics, Second Edition (Wiley-Liss, New York, 1999); Eckstein, editor, Oligonucleotides and Analogs: A Practical Approach (Oxford University Press, New York, 1991); Gait, editor, Oligonucleotide Synthesis: A Practical Approach (IRL Press, Oxford, 1984); Singleton, et al., Dictionary of Microbiology and Molecular biology, 2d ed., John Wiley and Sons, New York (1994), and Hale & Markham, the Harper Collins Dictionary of Biology, Harper Perennial, N.Y. (1991) and the like.

As used herein and in the appended claims, the singular forms “a” and “an” include plural referents unless the context clearly dictates otherwise. For example, the term “a protein” refers to one or more proteins, i.e., a single protein and multiple proteins.

Numeric ranges are inclusive of the numbers defining the range. All numbers should be understood to encompass the midpoint of the integer above and below the integer i.e., the number 2 encompasses 1.5-2.5. The number 2.5 encompasses 2.45-2.55 etc. When sample numerical values are provided, each alone may represent an intermediate value in a range of values and together may represent the extremes of a range unless specified. Percent ranges with only one end point (e.g., >90% or ≤10%) optionally include a second endpoint at the maximum or minimum percentage (e.g., ≥90% includes a range of 90%-100% and ≤10% includes a range of 0%-10%). Ranges (including percent ranges) with only one end point (e.g., ≥90 or ≤10) optionally include a second endpoint 10% higher or 10% lower than the provided endpoint (e.g., ≥90 includes a range of 90-99 and ≤10 includes a range of 1-10). Concentration percentages are w/v percentages unless otherwise indicated.

In the context of the present disclosure, “Argonaute” refers to an endonuclease that catalyzes cleavage of a single stranded nucleic acid or single strand of the double stranded nucleic acid governed by the sequence of a bound guide and may comprise (a)(i) an N-terminal domain (e.g., facilitating release of a target nucleic acid after cleavage) and a PAZ domain (e.g., which may hold the 3′ end of the guide pending hybridization of the guide with a complimentary sequence) or (ii) an effector domain and an APAZ domain, (b) a MID domain (e.g., which binds a short, single-stranded oligonucleotide guide), and/or (c) a PIWI domain (e.g., having a metal-dependent, RNase H-like endonuclease with activity conditioned on whether the PAZ domain is bound to the 3′ end of the guide and/or whether the guide is hybridized to a complementary target sequence). In some embodiments, an Argonaute may be a naturally occurring protein. In some embodiments, an Argonaute may be a non-naturally occurring protein. An Argonaute may have an amino acid sequence having at least 80%, at least 90%, at least 95%, or 100% sequence identity to a wild type Argonaute polypeptide (e.g., Argonaute from Thermus thermophilus). Examples of Argonautes include, without limitation, Argonautes from Aquifex aeolicus (AaeAgo, e.g., WP_010880937.1), Chlostridium perfringens (CpeAgo, e.g., WP_283721184.1), Mucilaginibacter paludis (MpaAgo, e.g., WP_008504757.1), Bacteroidetes bacterium (BbAgo, e.g.., MCA0383648.1), Chitinophaga costaii (CcAgo, e.g., WP_089708808.1), Chitinophagaceae bacterium (ChbAgo, e.g., MBL03353380.1). Some eukaryotic and prokaryotic Argonautes (e.g., MpaAgo, hAgo2, CpeAgo) are capable of binding unphosphorylated 5′OH and 5′-phosphorylated guides.

Argonautes include eukaryotic (e.g., mouse AGO2) and prokaryotic Argonautes. For example, Argonautes include eukaryotic AGO Argonautes, eukaryotic PIWI Argonautes, prokaryotic long A Argonautes, prokaryotic long B Argonautes, and prokaryotic short Argonautes. Argonaute may comprise an amino acid change relative to a reference sequence (e.g., a naturally occurring sequence) such as a deletion, insertion, substitution, variant, mutation, fusion, chimera, or any combination thereof. Argonaute refers to any modified (e.g., shortened, mutated, lengthened) polypeptide sequence or homologue of the reference (e.g., wild type) Argonaute. An Argonaute can be enzymatically inactive, partially active, constitutively active, fully active, inducibly active and/or more active, (e.g., more than the wild type homologue of the protein or polypeptide). A “thermostable” Argonaute is a protein that remains catalytically active for at least 5 minutes (e.g., at least 10 minutes) at elevated temperatures such as above 45° C., 50° C. or 55° C. An Argonaute catalytically active at physiological temperatures (e.g., 25-45° C.) may be referred to as a “mesophilic Argonaute”. With its guide bound to a complementary target sequence, an Argonaute creates a break in the phosphodiester backbone of the complementary target nucleic acid. In the case of double-stranded substrates, a break is only created in the strand which is complementary to the guide nucleic acid. As disclosed herein, a break in the other strand may be introduced using a second

Argonaute with a second guide.

An Argonaute may operably bind a guide to form an Argonaute: guide complex. An Argonaute: guide complex may be operable to specifically cut a substrate RNA (e.g., long RNA) at or near a substrate site having a sequence complementary to the guide. Argonautes may be useful for specific cleavage of RNA under reaction conditions that destabilize secondary structure of the target RNA without being so harsh as to degrade the target RNA. For example, non-thermophilic Argonautes including those disclosed herein may provide specific RNA cleavage whereas thermophilic Argonautes may require reaction temperatures that, in combination with the magnesium often used or required in Argonaute reactions, may lead to non-enzymatic, non-specific degradation of the RNA substrate.

Without limiting any embodiment to any particular mechanism of action, Argonautes are not single-stranded binding proteins even if they may be regarded as binding RNA substrates ahead of cleavage and release of products.

In the context of the present disclosure, “buffer” and “buffering agent” refer to a chemical entity or composition that itself resists and, when present in a solution, allows such solution to resist changes in pH when such solution is contacted with a chemical entity or composition having a higher or lower pH (e.g., an acid or alkali). Examples of suitable non-naturally occurring buffering agents that may be used in disclosed compositions, kits, and methods include HEPES, MES, MOPS, TAPS, tricine, and Tris. Additional examples of suitable buffering agents that may be used in disclosed compositions, kits, and methods include ACES, ADA, BES, Bicine, CAPS, carbonic acid/bicarbonic acid, CHES, citric acid, DIPSO, EPPS, histidine, MOPSO, phosphoric acid, PIPES, POPSO, TAPS, TAPSO, and triethanolamine.

In the context of the present disclosure, “cleave” refers to a break introduced into the sugar-phosphate or corresponding structural polynucleotide backbone as a result of specific enzymatic activity directed by a complementary guide (e.g. breaking of a phosphodiester bond which exists between bases complementary to bases 10 and 11 of a polynucleotide guide).

In the context of the present disclosure, “container” refers to a human-made container. A container may comprise one or more walls (e.g., defining an interior volume) and optionally one or more openings. Containers comprising one or more openings may further comprise one or more closures (e.g., removable closures) for some or all such openings. A closure optionally may comprise an aperture or a septum, for example, to provide fluid communication with a volume of the container and a connected or inserted tube or syringe. Examples of containers include boxes, cartons, bottles, tubes (e.g., test tubes, microcentrifuge tubes), plates (e.g., 96-well, 384-well plates), vials, pipette tips, and ampules. Containers and/or closures may comprise any desired material including paper, plastics, glass, silicone, composites, metals, alloys, or combinations thereof. Containers and/or closures may comprise materials that are compostable, recyclable, and/or sustainable.

In the context of the present disclosure and with respect to an amino acid residue or a nucleotide base position, “corresponding to” refers to positions that lie across from one another when sequences are aligned, e.g., by the BLAST algorithm. An amino acid position in a functional or structural motif in one polymerase may correspond to a position within a functionally equivalent functional or structural motif in another polymerase.

In the context of the present disclosure, “fusion” refers to two or more polypeptides, subunits, or proteins covalently joined to one another (e.g., by a peptide bond). For example, a protein fusion may refer to a non-naturally occurring polypeptide comprising a protein of interest covalently joined to a second polypeptide. Examples of a second polypeptide include a reporter protein (e.g., a green fluorescent protein), a purification tag (e.g., a 6xHis or 8xHis tag), and expression tag, a polynucleotide binding protein, an enzyme, a conjugation tag (e.g., a SNAP® tag), and a peptide linker (e.g., a flexible linker, an inflexible linker, a cleavable linker). Unless otherwise disclosed, the protein of interest may be nearer to the N-terminal end or nearer to the C-terminal end than the second polypeptide to which it is joined. A fusion protein may have one or more heterologous domains added to the N-terminus, C-terminus, and or the middle portion of the protein. A fusion may comprise a non-naturally occurring combined polypeptide chain comprising two proteins or two protein domains joined directly to each other by a peptide bond or joined through a peptide linker. If two parts of a fusion protein are “heterologous”, they are not part of the same protein in its natural state. In some embodiments, a fusion may comprise an Argonaute covalently joined to a second polypeptide. In some embodiments, an Argonaute may include a fusion to a heterologous targeting sequence, a linker, an epitope tag, a detectable fusion partner, such as a fluorescent protein, β-galactosidase, luciferase and/or functionally similar peptides. Components of a fusion protein may be joined by one or more peptide bonds, disulfide linkages, and/or other covalent bonds.

In the context of the present disclosure, “duplex” and “double stranded” refer to any conformation of a polynucleotide in which two polynucleotide strands (e.g., separate molecules or spatially separated portions of a single molecule) comprise secondary structure elements form via intermolecular base complementation. For example, strands of a duplex may be arranged anti parallel to one another in a helix (e.g., A-form, B-form, Z-form) with complementary bases of each strand paired with one another (e.g., in Watson-Crick base pairs). Paired bases may be stacked relative to one another to permit pi electrons of the bases to be shared. A polynucleotide may have a homoduplex conformation (e.g., DNA: DNA or RNA: RNA) or a heteroduplex confirmation (e.g., DNA: RNA).

In the context of the present disclosure, “guide” refers to a single-stranded oligonucleotide (a) capable of binding (e.g., hybridizing to) a polynucleotide having a complimentary sequence, (b) capable of binding an Argonaute, and (c) comprising (i) at least 12 nucleotides (e.g., 12-35 nucleotides), (ii) ≥50% deoxyribonucleotides (e.g., ≥60%, ≥70%, ≥80%, ≥90%, or ≤100% deoxyribonucleotides), (iii) ≤50% ribonucleotides (e.g., ≤10%, ≤25%, ≤35%, ≤45%, ≤50% ribonucleotides), (iv) optionally, a phosphorylated 5′ end, (v) optionally, a nucleotide sugar modification, and (vi) optionally, a nucleotide substitution. In some embodiments, a guide may comprise a phosphorylated 5′ end or another chemical modification at its 5′ end. A guide may be engineered or synthetic with a sequence selected to complement a desired target sequence. A guide maybe capable of directing an Argonaute polypeptide: guide DNA complex to a target polynucleotide. A DNA guide may be an oligonucleotide or polynucleotide that is synthetic or from a natural source such as genomic DNA, cDNA, extrachromosomal DNA, microbial DNA or viral DNA (e.g., the natural source differing from the Argonaute such that the guide and Argonaute together form a non-naturally occurring combination). The guide DNA is generally single stranded when used with Argonaute although it may be derived from dsDNA. While RNA guides may be used, it will be recognized that RNA guides may be impractical where cost and/or stability considerations are important.

In some embodiments, a guide length suitable for Argonaute cleavage of dsDNA (e.g., in the presence of a helicase or single strand binding protein) may comprise at least 12 nucleotides, for example, having a size range of 12-60 nucleotides, 14-50 nucleotides, 15-40 nucleotides, 16-35 nucleotides, 15-24 nucleotides, or 16-21 nucleotides. In some embodiments, a guide DNA may be greater than 21 nucleotides or at least 24 nucleotides in length. In some embodiments, a guide may be 16-21 nucleotides in length (e.g., 16, 17, 18, 19, 20 or 21 nucleotides).

In some embodiments, a guide may comprise a nucleotide sugar modification or a nucleotide substitution. In some embodiments, a nucleotide sugar modification comprises a 2′ sugar modification and maybe selected from the group consisting of a 2′-O—CH₃, a 2′-F, and a 2′-MOE modification. In some embodiments, a nucleotide substitution comprises one selected from the group consisting of locked nucleic acid (LNA), an unlocked nucleic acid (UNA), deoxyuridine, pseudouridine, 5-methylcytosine, 2-aminopurine, 2,6-diaminopurine, deoxyinosine, 5-hydroxybutynl-2′-deoxyuridine, 8-aza-7-deazaguanosine, and 5-nitroindole. In some embodiments, a guide molecule comprises a sugar modification and a nucleotide substitution.

The nucleotide sequence of a guide may or may not be degenerate. For example, guides with degenerate sequences may be useful for targeting nucleic acid sequences that are not fully known and/or for targeting more than one variant in a population of polynucleotides.

In the context of the present disclosure, “helicase” refers to a motor protein that moves linearly along double stranded nucleic acids unwinding or otherwise separating the component strands along base paired nucleosides. A helicase may or may not form a ring structure surrounding a nucleic acid substrate. A helicase may or may not unwind molecules that comprise partially single stranded nucleic acids (“α helicases ”). Examples of helicases include, without limitation, RecQ-family helicases (e.g., EcoRecQ DNA helicase from Escherichia coli (WP_096324295.1), CpeRecQ from Clostridium perfringens (WP_011590145.1), CbuRecQ from Clostridium butyricum (WP_003411240.1)); DNA helicases from T4-like bacteriophages (e.g., T4 gp41, T4 gp41 associated with T4 gp59, T4 UvsW, T4 Dda and Slur07 Dda); T7 bacteriophage gp4 DNA helicase; RecBCD-family helicases (e.g., E. coli RecBCD DNA helicase); modified RecBCD helicases (e.g., RecB^{exo-}helicase, RecB^{exo-}C, RecB^{exo-}CD, RecΔB, RecΔBC, RecΔBCD); UvrD/PcrA family helicases, e.g., E.coli EcoUvrD, E. coli Rep, M. tuberculosis PerA, M. leprae PerA; and/or E. coli Tra helicase. A helicase may unwind, for example, linear, nicked circular, and/or supercoiled circular DNA.

In the context of the present disclosure, “immobilized” refers to covalent attachment of an enzyme to a solid support with or without a linker. Examples of solid supports include beads (e.g., magnetic, agarose, polystyrene, polyacrylamide, chitin). Beads may include one or more surface modifications (e.g., O⁶-benzyleguanine, polyethylene glycol) that facilitate covalent attachment and/or activity of an enzyme of interest. For example, a support may comprise a ligand and an enzyme may have a receptor for such ligand, or an enzyme may comprise a ligand and a support may comprise a receptor for such ligand. Receptor-ligand binding may be covalent or non-covalent. Non-covalent attachment (e.g., avidin: biotin, chitin: CBP) may be useful in some embodiments, for example, where the level of dissociation of the binding partner is deemed tolerable. A linker may be disposed between a support and an enzyme. For example, linker disposed between a support and an enzyme may have a first covalent bond to the support and a second covalent bond to the enzyme. An immobilized enzyme comprising a ligand- receptor attachment may have a linker disposed between the support and the ligand-receptor attachment, a linker disposed between the enzyme and the ligand-receptor attachment, or both. An immobilized enzyme comprising a linker may also comprise an optional covalent bond directly between the enzyme and the support. A linker may be of any desired length and have any desired range of motion. A peptide linker may comprise one or more repeats (e.g., 1-10 repeats) of glycine-serine.

In the context of the present disclosure, “modified nucleotide” refers to nucleotides having a modification on the sugars (e.g., 2′-fluororibose, ribose, 2′-deoxyribose, arabinose, and hexose); and/or in the phosphate groups (e.g., phosphorothioates and 5′-N-phosphoramidite linkages); and/or in the nucleotide base (e.g., as described in U.S. Pat. No. 8,383,340; WO 2013/151666; U.S. Pat. No. 9,428,535 B2; US 2016/0032316). Examples of modified nucleotides include pseudouridine and N1-methyl-pseudouridine.

In the context of the present disclosure, “non-naturally occurring” refers to a molecule (e.g., a polynucleotide, polypeptide, carbohydrate, or lipid) or composition that does not exist in nature. Such a molecule or composition may differ from naturally occurring molecules or compositions in one or more respects. For example, a polymer (e.g., a polynucleotide, polypeptide, or carbohydrate) may differ in the kind and arrangement of the component parts (e.g., nucleotide sequence, amino acid sequence, or sugar molecules). A polymer may differ from a naturally occurring polymer with respect to the molecule(s) to which it is linked. For example, a “non-naturally occurring” polypeptide (e.g., protein) may differ from naturally occurring polypeptides in its secondary, tertiary, or quaternary structure, by having (or lacking) a chemical bond (e.g., a covalent bond including a peptide bond, a phosphate bond, a disulfide bond, an ester bond, and ether bond, and others) to a lipid, a carbohydrate, a second polypeptide (e.g., a fusion protein), or any other molecule. Similarly, a “non-naturally occurring” polynucleotide or nucleic acid may comprise (or lack) one or more other modifications (e.g., an added label or other moiety) to the 5′-end, the 3′ end, and/or between the 5′-and 3′-ends (e.g., methylation) of the nucleic acid. A “non-naturally occurring” molecule or composition may differ from naturally occurring compositions in one or more of the following respects: (a) having components that are not combined in nature, (b) having components in ratios and/or concentrations not found in nature, (c) lacking one or more components otherwise found in naturally occurring molecules or compositions (e.g., a cell-free composition, a chromosome-free composition, a histone-free composition, a polymerase-free composition, a cell membrane-free composition), (d) having a form not found in nature (e.g., dried, freeze dried, lyophilized, crystalline, aqueous, immobilized), and (e) having one or more additional components beyond those found in nature (e.g., a buffering agent, a detergent, a dye, a solvent or a preservative).

In the context of the present disclosure, “oligonucleotide” refers to a polymer of nucleotides comprising naturally occurring nucleotides, non-naturally occurring nucleotides, derivatized nucleotides, or a combination thereof. As used herein, the term “complementarity” refers to the ability of nucleotides, or analogues thereof, to form Watson-Crick base pairs. Complementary nucleotide sequences will form Watson-Crick base pairs and non-complementary nucleotide sequences will not.

With reference to an amino acid, “position” refers to the place such amino acid occupies in the primary sequence of a peptide or polypeptide numbered from its amino terminus to its carboxy terminus. A position in one primary sequence may correspond to a position in a second primary sequence, for example, where the two positions are opposite one another when the two primary sequences are aligned using an alignment algorithm (e.g., BLAST (Journal of Molecular Biology. 215(3): 403-410) using default parameters (e.g., expect threshold 0.05, word size 3, max matches in a query range 0, matrix BLOSUM62, Gap existence 11 extension 1, and conditional compositional score matrix adjustment) or custom parameters). An amino acid position in one sequence may correspond to a position within a functionally equivalent motif or structural motif that may be identified within one or more other sequence(s) in a database by alignment of the motifs.

In the context of the present disclosure, “programmed” including “programmed to cleave”, with reference to an Argonaute, refers to an Argonaute comprising or bound to a sequence-specific guide, the sequence of which is complementary to a target sequence such that, when the guide and the target are hybridized, the Argonaute is capable of cutting the target within or near the target sequence.

In the context of the present disclosure, “single-stranded” refers to an individual polynucleotide molecule that is free of secondary structure arising from interbase intermolulcular hydrogen bonds (hydrogen bonds between a base of such polynucleotide molecule and a base of any other (separate) polynucleotide molecule). For example, a single-stranded polynucleotide (e.g., a ssRNA) may be free of sequence-specific hydrogen bonds, free of Watson-Crick hydrogen bonds, free of noncannonical hydrogen bonds or free of any two of up to all three of the foregoing hydrogen bonds. A ssRNA may be free of hydrogen bonds (intermolecular and intramolecular) within 10, 20, 30, 40, and/or 50 bases of an Argonaute cleavage site. A single-stranded polynucleotide (e.g., a ssRNA) may be linear or circular. For clarity, a single-stranded polynucleotide molecule may exclude or include intramolecular hydrogen bonds. Intramolecular interbase hydrogen bonds, if present in a single stranded polynucleotide, may be associated with secondary structures that resemble separate polynucleotides annealed into a double-strand.

In the context of the present disclosure, “single-stranded DNA binding protein” and “single-stranded binding protein” refer to a protein that binds to ssDNA. The genomes of most organisms, including bacteria (e.g., E. coli), viruses (e.g., herpes viruses) and mammals, encode at least one SSB. SSBs of interest include, but are not limited to, ET SSB, E. coli recA, T7 gene 2.5 product (gp2.5), T4 gene 32 product (gp32), E. coli SSB, replication protein A (RPA) from archaeal and eukaryotic organisms, Nanoarchaeum equitans SSB-like protein, UvrD, RadA, Rad51, phage lambda RedB or Rac prophage RecT. An SSB may be thermostable or mesolabile. An SSB may have at least 80%, at least 90%, at least 95%, or 100% sequence identity to a wild type SSB. For clarity, single-stranded binding protein, in the context of the present disclosure, do not include Argonautes.

In the context of the present disclosure, “substitution” refers to an amino acid residue at a position in a comparator amino acid sequence that differs with respect to a corresponding position of a reference amino acid sequence, where the comparator and reference sequences are at least 60% identical to each other or at least 70% identical to each other or at least 80% identical to each other. A reference sequence and comparator sequence may have the same length or similar lengths (e.g., differing by ≤12%, ≤5%, ≤1%). A substitute amino acid residue at a position, in addition to differing from the corresponding position of a reference amino acid sequence, may differ from the amino acid at the corresponding position of all naturally occurring sequences that are at least 60% identical to each other or at least 70% identical to each other or at least 80% identical to the reference sequence. Optionally, a substitute amino acid may have different properties than the amino acid in the corresponding position of the reference sequence. Optionally, a substitute amino acid may have similar properties to the amino acid in the corresponding position of the reference sequence (a “conservative” substitution). For example, a non-polar amino acid (e.g., A, V, L, I, M, W, and F (and optionally C, G, and P) may substitute for another non-polar amino acid, a polar amino acid (e.g., N, Q, S, T, and Y) may substitute for another polar amino acid (e.g., C, D, E, H, K, N, P, Q, R, S, and T), a positively charged amino acid (H, K, and R) may substitute for another positively charged amino acid, and a negatively charged amino acid (e.g., D and E) may substitute for another negatively charged amino acid. A substitute amino acid may be a natural amino acid (e.g., replacing another natural amino acid or a non-natural amino acid). A substitute amino acid may be a non-natural amino acid (e.g., replacing a natural amino acid or another non-natural amino acid).

In the context of the present disclosure, “target” refers to a nucleic acid having a nucleic acid sequence, which may be a nonnaturally occurring sequence (e.g., a therapeutic RNA, a vaccine), a chromosomal sequence or an extrachromosomal sequence, (e.g., an episomal sequence, a minicircle sequence, a plasmid, a mitochondrial sequence, a chloroplast sequence, etc.). A target nucleic acid may be a ssRNA. For clarity, a target may include any RNA molecule the is free of interbase intermolulcular hydrogen bonds (hydrogen bonds between a base of such RNA and any other RNA molecule), even if such RNA molecule includes one or more intramolecular hydrogen bonds. For example, a target ssRNA may be free of sequence-specific hydrogen bonds, free of Watson-Crick hydrogen bonds, free of noncannonical hydrogen bonds or free of any two of up to all three of the foregoing hydrogen bonds. A target ssRNA may be free of hydrogen bonds (intermolecular and intramolecular) within 10, 20, 30, 40, and/or 50 bases of an Argonaute cleavage site. Secondary structure in a target (e.g., arising from hybridization of complementary bases) may contribute to the creation of higher order tertiary structures that may inhibit or enhance the activity of Argonautes or accessory proteins used in conjunction with Argonautes. A ssRNA may be linear or circular. A target RNA maybe a DNA-RNA chimera.

All publications, patents, and patent applications mentioned in this specification are herein incorporated by reference to the same extent as if each individual publication, patent, or patent application was specifically and individually indicated to be incorporated by reference. Reagents referenced in this disclosure may be made using available materials and techniques, obtained from the indicated source, and/or obtained from New England Biolabs, Inc. (Ipswich, MA).

### Compositions

The present disclosure provides, in some embodiments, compositions for producing a break in target RNA (e.g., linear RNA, circular RNA). In some embodiments, compositions may produce a break in single-stranded RNA at temperatures from, for example, 4° C. to 50° C. (e.g., 4° C. to 20° C., 10° C. to 25° C., 15° C. to 30° C., or 20° C. to 35° C., 25° C. to 40° C., 30° C. to 45° C., 35° C. to 50° C., or 40° C. to 50° C.). A composition may comprise, for example, an Argonaute and a guide, optionally wherein the Argonaute and guide are operatively bound to one another. In this context, the terms bound and operatively bound refer to the capacity of the combined Argonaute: guide complex to bind other polynucleotides (e.g., RNA) having a sequence complementary to at least a portion of the guide sequence guide and endonucleolytically cleave such other polynucleotide within or proximal to such complementary sequence. A composition may comprise, for example, an Argonaute bound to a guide, and optionally, a target RNA. In some embodiments, a single Argonaute with a single guide may produce a single-stranded break in a target RNA (e.g., a target RNA comprising a sequence complementary to at least a portion of the guide sequence). Single-stranded DNA with longer and/or more complex repeating sequences may also be cleaved with an Argonaute bound to a single guide sequence.

Compositions, in some embodiments, may exclude whole cells and/or exclude cell extracts. For example, where a composition is configured to cleave a target polynucleotide, it may be desirable or required to exclude cells and cell extracts that may interfere. Compositions may exclude, for example, enzymes or other materials that may nick a strand of (e.g., nicking enzymes) or cleave (e.g., single-stranded and double-stranded nucleases) polynucleotides or that ligate (e.g., ligases) polynucleotides cut by Argonaute/guide complexes. Compositions may lack, according to some embodiments, one or more of operable cell membranes, ribosomes, nucleus, cytoplasm, mitochondria, and/or a cell wall.

In some embodiments, a composition may include or exclude a helicase and may include or exclude a single-stranded DNA binding protein. A composition, according to some embodiments, may include or exclude components beyond an Argonaute and a guide. For example, a composition may include one or more polynucleotides that are actual or potential substrates for programmed cleavage (e.g., any ssRNA), one or more transcription substrates (and a polymerase) to produce the target RNA (e.g., plasmids, phage, vectors, genomic DNA, organellar DNA, library DNA), detection agents, nucleotide triphosphates (e.g., ATP, GTP, CTP, TTP or modified versions thereof), buffers, salts, detergents, and/or crowding agents. A composition may include one or more other proteins, examples of which include polymerases, ligases, nucleases, helicase loading proteins (e.g., T4 gp59 protein for T4 gp41 helicase or MutL protein for EcoUvrD helicase), and/or helicase processivity enhancers (e.g., RepD for PcrA helicase).

In some embodiments, off-target activity may be reduced or absent from compositions and methods of the disclosure. Selection of the length, complexity, and/or G: C content of the guide sequence(s), for example, may result in compositions and methods for cleaving RNA with little or no off-target activity.

### Methods

The present disclosure relates, in some embodiments, to methods for cleaving an RNA of interest (e.g., a target RNA). An RNA of interest may be any RNA molecule including, for example, nonnaturally occurring RNA, viral RNA, prokaryotic RNA, eukaryotic RNA, and/or archaeal RNA. An RNA of interest may be a messenger RNA (mRNA), ribosomal RNA (rRNA), transfer RNA (tRNA), small RNA (sRNA), snoRNA, snRNA, microRNA (miRNA), long noncoding RNA (IncRNA), circular RNA (circRNA), aptamer RNA, antisense RNA, silencing RNA (siRNA), guide RNA (gRNA), or any combination thereof. An RNA of interest may be itself a therapeutic RNA or may be included in a therapeutic RNA composition.

In the context of the present disclosure, “IVT template” refers to an RNA or DNA molecule that may be transcribed in vitro using any available in vitro transcription system including, for example, a cell lysate or a HiScribe® T7 high yield RNA synthesis kit (NEB, Inc.) An IVT template may be a single-stranded RNA comprising, in a 5′ to 3′ direction, a 5′ untranslated region (“UTR”), a coding sequence (e.g., defined by start and stop codons), a 3′ UTR, and/or a poly(A) tail. An RNA molecule may serve as an IVT template, for example, when combined with an RNA-dependent RNA polymerase.

The present disclosure relates, in some embodiments, to methods for cleaving a single-stranded RNA. Cleavage may be selective, for example, in that a single-stranded target having an identical sequence to a duplex molecule at the same enzyme to target ratio and like conditions will result in more cleavage product (more complete digestion) than the duplex molecule. In some embodiments, a method may comprise contacting an Argonaute bound to a suitable guide (the resulting complex may be referenced as an Argonaute: guide) and a a single-stranded RNA, the Argonaute having an amino acid sequence that is at least 90% identical (e.g., at least 95% identical) to any of Argonautes disclosed herein to produce cleavage products. A single-stranded RNA may comprise at least one targeted sequence complementary to at least a portion of the guide. In some embodiments, a method may comprise contacting a single-stranded RNA comprising at least one targeted sequence complementary to at least a portion of a guide of a Argonaute: guide complex to produce the cleavage products. Cleavage products may comprise one or more fragments of the single-stranded RNA. A single-stranded RNA may be a long RNA (e.g., at least 5000 nucleotides) and/or may comprise multiple (e.g., >5) sequences of interest separated by guide targeted sequences.

According to some embodiments, a single-stranded RNA may comprise one or more modified nucleotides (e.g., anywhere in the nucleotide sequence including within the recognition sequence). Examples of modified nucleotides include N⁶-methyl-adenosine (m⁶A), 1-methyl-adenosine (m¹A), 5-methyl-cytidine (m⁵C), 5-hydroxymethyl-cytidine (hm⁵C),N⁴-acetyl-cytidine (ac⁴C), 5-methoxycytidine (mo⁵C), 4-thiouridine (S⁴U), 2-thiouridine (S²U), pseudouridine (ψ), N¹-methyl-pseudouridine (m¹ψ), 5-methyluridine (m⁵U), or 5-methoxyuridine (mo⁵U). Examples of single-stranded RNA include RNA molecules selected from (or RNA comprising RNA molecules selected from a messenger RNA (mRNA), a ribosomal RNA (rRNA), a transfer RNA (tRNA), a small RNA (sRNA), a microRNA (miRNA), a long noncoding RNA (IncRNA), a circular RNA (circRNA), a transfer RNA (tRNA), an aptamer RNA, an antisense RNA, a silencing RNA (siRNA), a guide RNA (gRNA), or a therapeutic RNA. In some embodiments, a method may comprise contacting an Argonaute: guide and a polycistronic single-stranded RNA comprising at least ten guide targeted sequences that each comprises a sequence complementary to at least a portion the guide of the Argonaute: guide to produce cleavage products. Cleavage products (e.g., cleavage products of a polycistronic template) may comprise at least one IVT template. In some embodiments, a method comprises contacting a single-stranded RNA with a first Argonaute: guide and further comprises contacting the single-stranded RNA with a second Argonaute: guide (e.g., concurrently) wherein the single-stranded RNA comprises at least one targeted guide sequence that corresponds to each of the first and second Argonaute: guides.

In some embodiments, a method may further comprise analyzing at least one cleavage product. Analyzing a cleavage product may comprise analyzing the cleavage product by electrophoretic, photometric, and/or mass spectrometric analysis.

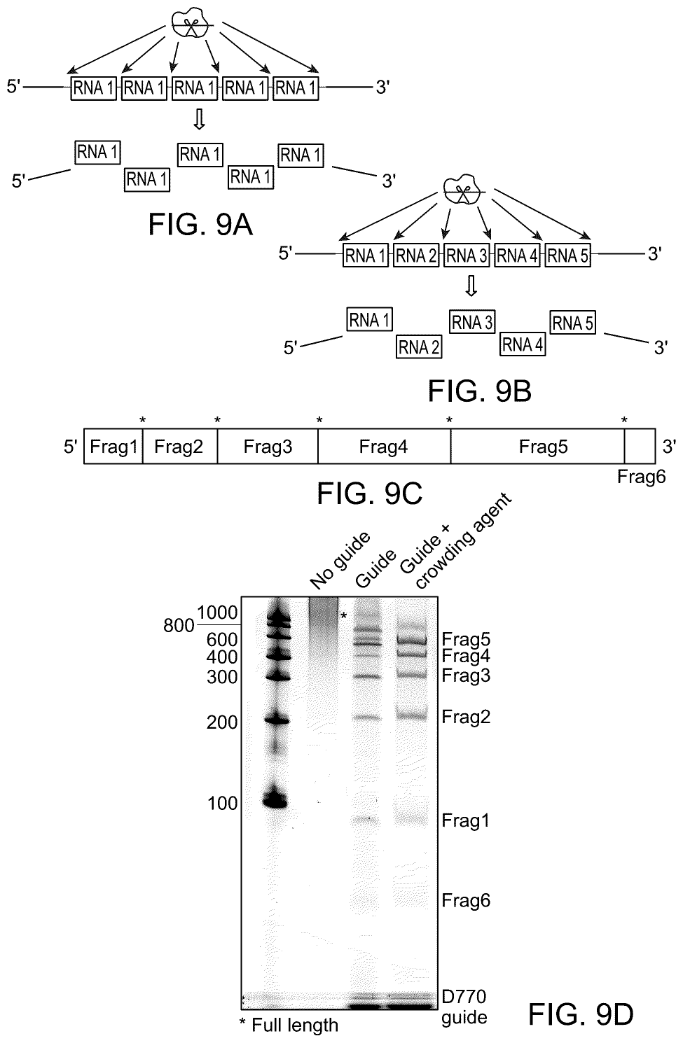

In the context of the present disclosure, “polycistronic template” refers to an RNA (or DNA) molecule comprising, in a 5′ to 3′ direction, E(CS-GRS)ₙ(CS)ₘ(T)ₓ3′, wherein E is a 5′ end portion, CS encodes a coding sequence, GRS encodes an targeted guide recognition sequence, n=≥1, ≥2, ≥3, ≥4, ≥5, ≥10, ≥15, ≥20, ≥25, or ≥50, m=0 or 1, T encodes a poly(A) tail, x=0 or 1, and 3′ represents the 3′ end of the polycistronic template molecule. For clarity, if n=1, then m≠0. Transcription of a polycistronic template (e.g., a DNA template) may produce a polycistronic single-stranded RNA comprising, in a 5′ to 3′ direction, (cs-grs)ₙ(cs)ₘ(A)ₓ3′, wherein cs encodes a coding sequence, grs encodes an targeted guide recognition sequence, A is a poly(A) tail, and 3′ represents the 3′ end of the polycistronic template molecule (with m, n, and x as defined above). In some embodiments, coding sequences in a polycistronic RNA may be the same (e.g., FIG. 9A) or different (e.g., FIG. 9B). In some embodiments, targeted guide sequences of a polycistronic RNA may be the same or different.

Polycistronic RNAs may be produced efficiently from polycistronic templates, for example, by performing in vitro transcription with a viral RNA polymerase (e.g., T7 RNA polymerase, SP6 RNA polymerase, T3 RNA polymerase). In some embodiments, a method may include transcribing a polycistronic template to produce a polycistronic RNA and contacting the polycistronic RNA with an Argonaute: guide (e.g., compatible with targeted guide sequences in the polycistronic RNA) to produce Argonaute cleavage products comprising one or more (copies of) RNA molecules of interest (e.g., each comprising an IVT template).

In some embodiments, a polycistronic template may be configured to encode ≥1, ≥2, ≥3, 4, ≥5, ≥10, ≥15, >20, ≥25, or ≥50 different RNA species and a sequence specific targeted guide site (optionally, wherein each guide recognition site may be the same or different) positioned between each of the different RNA species. In some embodiments, a method may comprise contacting a polycistronic template (the polycistronic template having sequences encoding two or more different coding sequences) with an RNA polymerase (e.g., a viral RNA polymerase or variant thereof) to form transcription products comprising polycistronic RNA (the polycistronic template having two or more different coding sequences) and contacting the transcription products with one or more Argonaute: guides (the one or more Argonaute: guides compatible with targeted guide sequences in the polycistronic RNA) to produce Argonaute cleavage products comprising separate RNA molecules, each having one of the different species of coding sequences. An example of such a method is illustrated in FIG. 9B.

### Kits

The present disclosure further relates to kits including an Argonaute, a buffer, and optionally one or more other components. For example, a kit may include an Argonaute and one or more of a guide, dNTPs, rNTPs, primers, other enzymes (e.g., polymerases, methylases, capping enzymes, helicases, other enzymes), buffering agents, or combinations thereof. Enzymes may be included in a storage buffer. Any suitable storage buffer may be used, for example, buffers comprising one or more of a cryoprotectant (e.g., a polyol such as glycerol, an antifreeze protein), a salt, a detergent, a reducing agent, a sugar, a chelator, and an antimicrobial agent and having a pH tolerated by the enzyme to be stored, for example, between pH 6 and 9. A composition or kit may include a reaction buffer which may be in concentrated form, and the buffer may contain additives (e.g. glycerol), salt (e.g. NaCl, KCl), reducing agent, EDTA or detergents, among others. Detergents include nonionic detergents (e.g., t-octylphenoxypolyethoxyethanol), anionic detergents (e.g., alkylbenzene sulfonates), cationic detergents (e.g., alkylbenzene quaternary ammonium), and zwitterionic detergents. A composition or kit comprising dNTPs may include one, two, three of all four of dATP, dTTP, dGTP and dCTP. A kit comprising rNTPs may include one, two, three of all four of rATP, rUTP, rGTP and rCTP. A kit may further comprise one or more modified nucleotides. A kit may optionally comprise one or more primers (random primers, bump primers, exonuclease-resistant primers, chemically-modified primers, custom sequence primers, or combinations thereof). A kit may optionally comprise chaotropic reagents such as formamide or urea that may disrupts the secondary and/or tertiary structure of target RNA or DNA guide.

A reagent kit may be a non-natural collection of components configured, for example, for convenient storage, shipping, delivery, and/or use. A kit may include one or more containers, each comprising one or more materials and each combinable with one or more kit components or other materials to form one or more of the compositions disclosed herein and/or to perform one or more methods disclosed herein. One or more components of a kit may be included in one container for a single step reaction, or one or more components may be contained in one container, but separated from other components for sequential use or parallel use or controlable commencement of a desired condition or reaction. The contents of a kit may be formulated for use in a desired method or process. At least one container of a kit may exclude whole cells and/or exclude cell extracts.

A kit is provided that contains: (i) an Argonaute; and (ii) a buffer. The Argonaute may have a lyophilized form or may be included in a buffer (e.g., a storage buffer or a reaction buffer in concentrated form). A kit may contain the Argonaute in a mastermix suitable for receiving and amplifying a template nucleic acid. Argonaute may be a purified enzyme so as to contain substantially no DNA or RNA and no nucleases. The reaction buffer in (ii) and/or storage buffers containing the DNA polymerase in (i) may include non-ionic, ionic e.g. anionic or zwitterionic surfactants and crowding agents. A kit may include the Argonaute and the reaction buffer in a single tube or in different tubes.

A subject kit may further include instructions for using the components of the kit to practice a desired method. The instructions may be recorded on a suitable recording medium. For example, instructions may be printed on a substrate, such as paper or plastic, etc. As such, the instructions may be present in the kits as a package insert, in the labeling of the container of the kit or components thereof (i.e., associated with the packaging or subpackaging) etc. Instructions may be present as an electronic storage data file residing on a suitable computer readable storage medium (e.g. a CD-ROM, a flash drive). Instructions may be provided remotely using, for example, cloud or internet resources with a link or other access instructions provided in or with a kit.

### EXAMPLES

Some specific example embodiments may be illustrated by one or more of the examples provided herein.

### Example 1: Multiplexed Programmed Cleavage of Messenger RNA (mRNA)

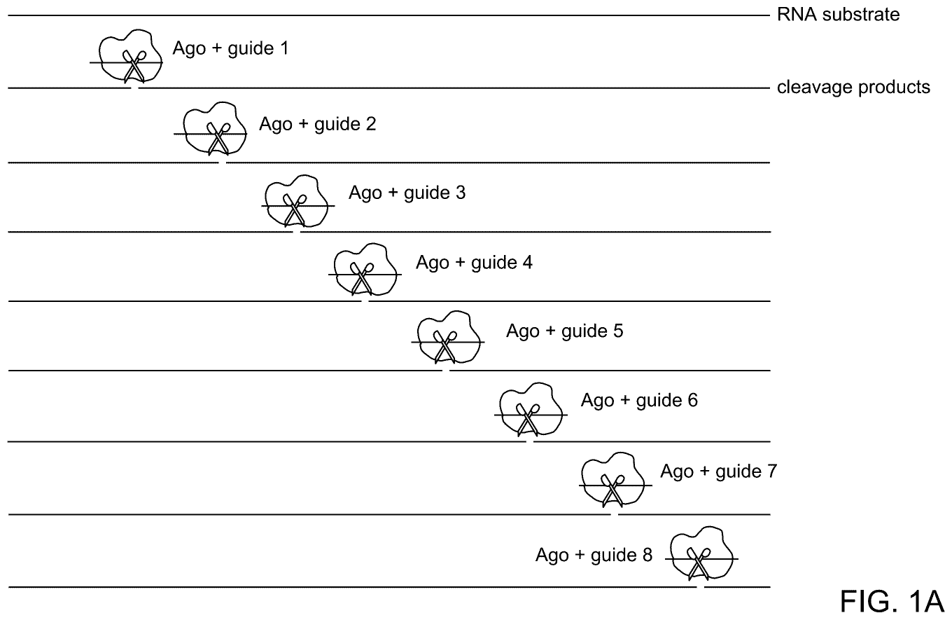

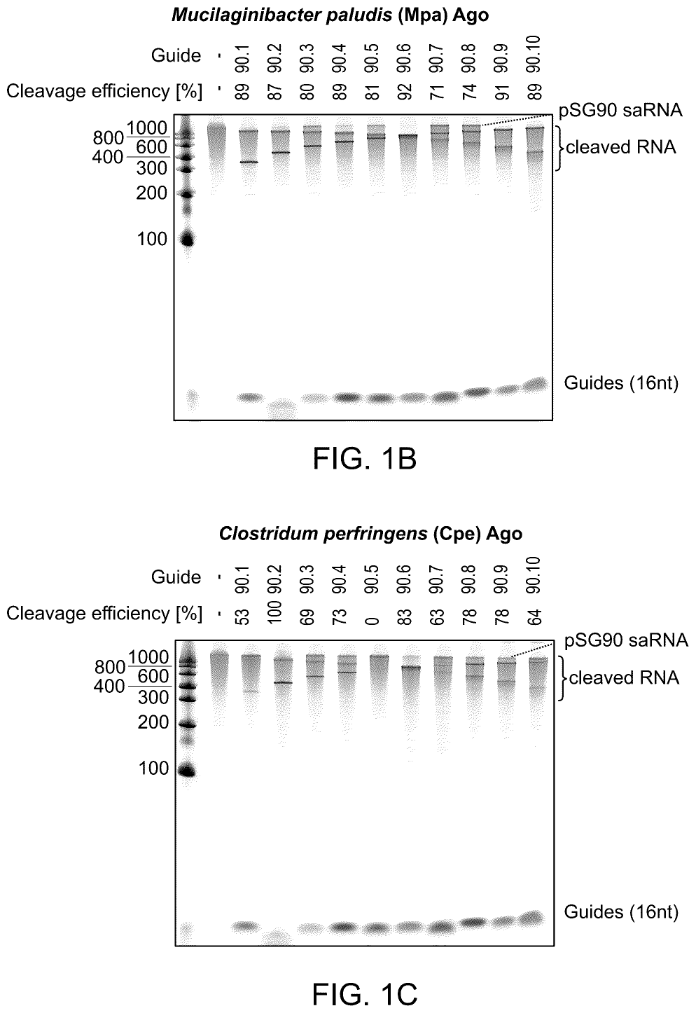

FIG. 1A shows hypothetical reactions containing a target RNA that is treated with an Ago and a single unique guide (G1-G8) per reaction, each reaction (R1-R8) resulting in the cleavage of the target RNA into two distinct cleavage fragments whose lengths are determined by the guide-programmed cleavage site. Each guide binds a complementary sequence in the target RNA with the complementary sequences positioned at roughly evenly space sites along the target RNA molecule to produce roughly evenly spaced cleavage sites. The two cleavage products are expected to have very different sizes when the complementary sequences and cleavage sites are positioned toward the 5′ or 3′ ends (e.g., G1 or G8) with progressively less pronounced size differences as complementary sequences approach the middle of the target RNA.

This expected pattern was observed in example test reactions. Specifically, in 10 μl reaction volume, 0.37 pmol of a 1678 nt mRNA (pSG90 saRNA; SEQ ID 1) were incubated in 1× ThermoPol reaction buffer (NEB #B9004S) with either 1.85 pmol Mucilaginibacter paludis (Mpa) or Clostridum perfringens (Cpe) Ago with or without 9.25 pmol of a unique DNA guide (16 nt; SEQ ID NOS: 2-11) at 50° C. in a Thermocycler for 30 min (ratios: substrate:Ago=1:5, Ago:guide=1:5). The reactions were stopped by the addition of 0.08 units (U) NEB proteinase K (NEB#P8107S) and incubation at room temperature for 5 min. The samples were denatured (4 min at 70° C.) in 1× RNA loading dye (NEB #B0363S) and analyzed on 6% Novex™ TBE-Urea gels (ThermoFisher #EC6865BOX). Results of reactions with the MpaAgo are shown in FIG. 1B. Results of reactions with CpeAgo are shown in FIG. 1C. The percentage of cleaved substrates by both Agos is shown in the space above each lane.

The guide sequences were chosen to generate site specific cleavages ˜100 nt apart from each other in the pSG90 mRNA, and 72 nt apart from each other in the FLuc mRNA. The data show high specificity (shown by the lack of off-target/unwanted cleavage products) and efficiency of cleavage (cleavage of between 71% and 91% of the starting RNA) for both enzymes.

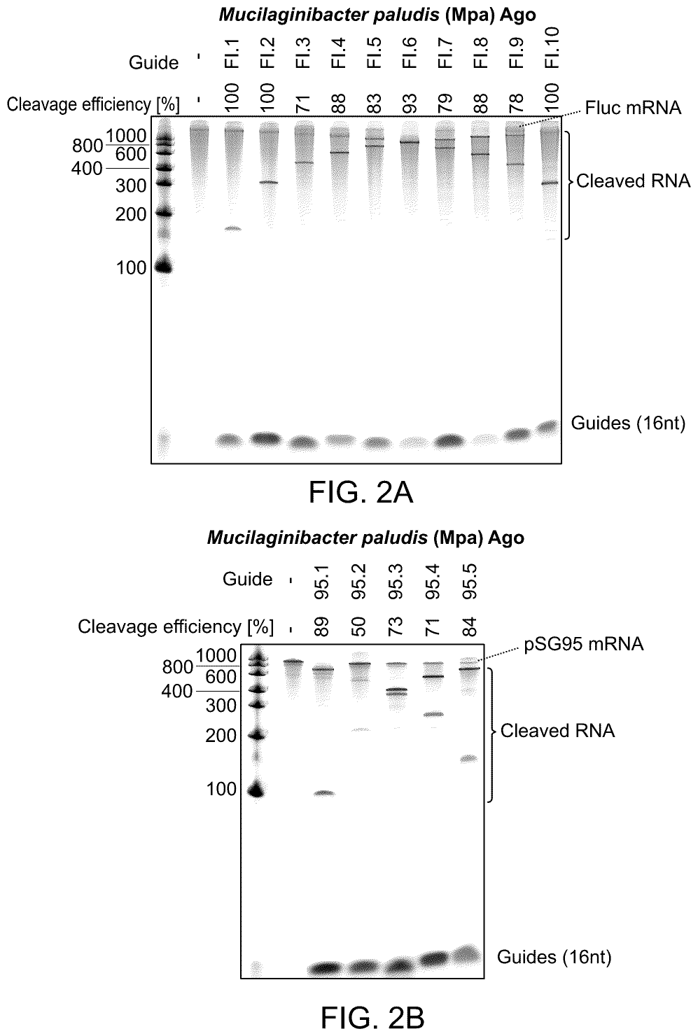

To confirm the specificity of guide programmed pAgos on other RNA substrates, 0.33 pmol of FLuc mRNA (SEQ ID 12) were incubated with 1.65 pmol MpaAgo and 9.25 pmol DNA guides (16 nt, SEQ ID NOS: 13-22), targeting unique sites in the FLuc mRNA approximately 72 nt apart from each other (FIG. 2A). Additionally, 0.86 pmol of a 723 nt mRNA (pSG95 epo, SEQ ID 23) were reacted with 4.31 pmol MpaAgo and 21.53 pmol unique DNA guides (16 nt; SEQ ID NOS: 24-28) that were programmed to cleave approximately 100 nt apart from each other (FIG. 2B). These reactions contained the same enzyme:guide:substrate ratios and followed the same experimental procedures as described above. In both cases, the expected cleavage fragments were found, again showing a highly specific (no background/off-target cleavage) and efficient cleavage of the substrates by the Ago with its respective guides. The percentage of cleaved substrates by each Ago is shown in the space above each lane.

To further confirm the specificity of the cleavage by a guide-programmed Ago, a 5-fold upscaled reaction containing 1.85 pmol pSG90 saRNA (SEQ ID 1), 9.25 pmol MpaAgo, and 46.25 pmol guide 90.2 (SEQ ID 3) was reacted for 30 min at 50° C. in a 50 μl reaction. 0.12 U of thermolabile proteinase K (NEB #P8111S) and 40 U of murine RNase inhibitor (NEB #M0314S) where added and the reactions incubated for 15 min at 37° C., followed by an incubation for 10 min at 55° C. to heat inactivate the proteinase K. To prepare the cleavage products for ONT nanopore sequencing, the reactions were treated with T4 Polynucleotide Kinase (T4 PNK; NEB #M0201S). In a 60 μl reaction, 46 μl of above's reaction were incubated with 40 U murine RNase inhibitor and 10 U T4 PNK in T4 PNK reaction buffer for 30 min at 37° C., and subsequently column purified (Zymo #R1013). The purified RNA was subjected to poly(A) tailing in a 20 μl reaction containing 15 μl purified RNA, 0.5 mM ATP, and 5 U E. coli poly(A) polymerase (NEB #M0276S) for 1.5 min at 37° C., and column purified. A ONT nanopore library was prepared as described in the ONT Direct RNA sequencing RNA002-DRS 9080 v2 manual. Cleavage sites were detected and mapped to the respective substrate RNA, showing that most of the cleavage occurred at the programmed site at position 465 (67%), with significantly less cleavage at adjacent locations (463, 26% and 464, 7%; TABLE 1) under the specific conditions tested here.

TABLE 1
ONT nanopore detection of the cleavage site (vertical bar “I”) in pSG90 saRNA by
MpaAgo programmed with guide 90.2.

| substrate | position | num ends | cleavage site reads | NTS 454-475 of SEQ ID NO: 1 |
|---|---|---|---|---|
| pSG90 saRNA | 465 | 72962 | 67.0% | CACCAUCGCUCU\|GCUUUCACAA |
| pSG90 saRNA | 464 | 7489 | 6.9% | CACCAUCGCUC\|UGCUUUCACAA |
| pSG90 saRNA | 463 | 28435 | 26.1% | CACCAUCGCU\|CUGCUUUCACAA |

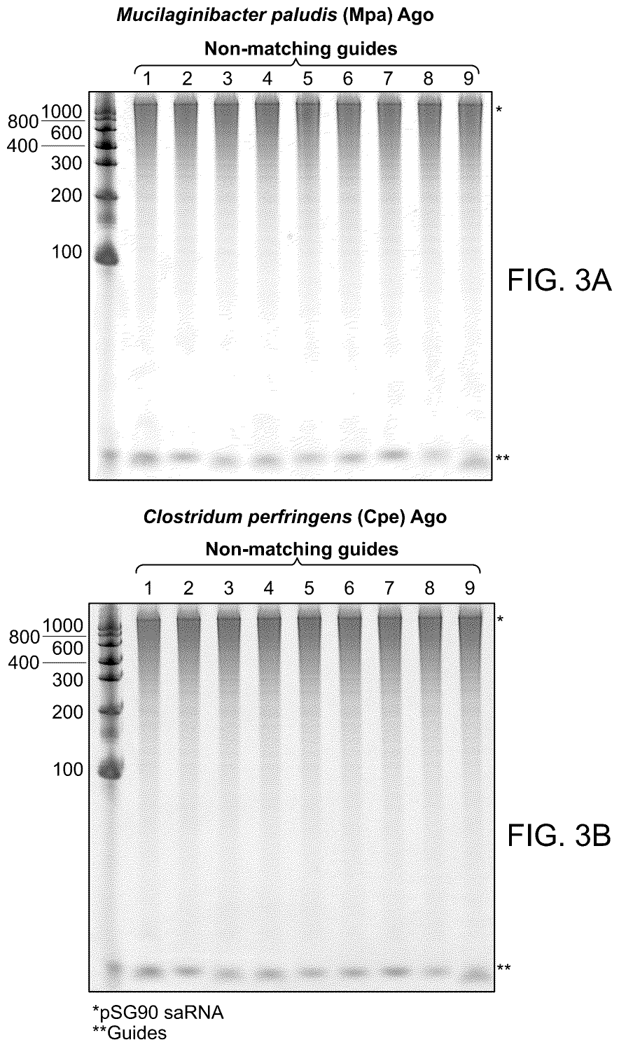

To further confirm the specificity of the guide-programmed Agos, 200 ng (0.37 pmol) of pSG90 saRNA were incubated in 1× ThermoPol reaction buffer with either 1.85 pmol MpaAgo (FIG. 3A) or CpeAgo (FIG. 3B) with 9.25 pmol of nine unique, but non-matching DNA guides at 50° C. in a Thermocycler for 30 min. The reactions were stopped by the addition of 1 μl 1:10 diluted NEB proteinase K and incubation at room temperature for 5 min. The samples were denatured (4 min at 70° C.) in 1× RNA loading dye and analyzed on 6% Novex™ TBE-Urea gels. As expected, the combination of Mpa or CpeAgo with guides whose sequences did not match a sequence in the RNA substrate showed no substrate cleavage, suggesting a guide-dependent and -induced high specificity.

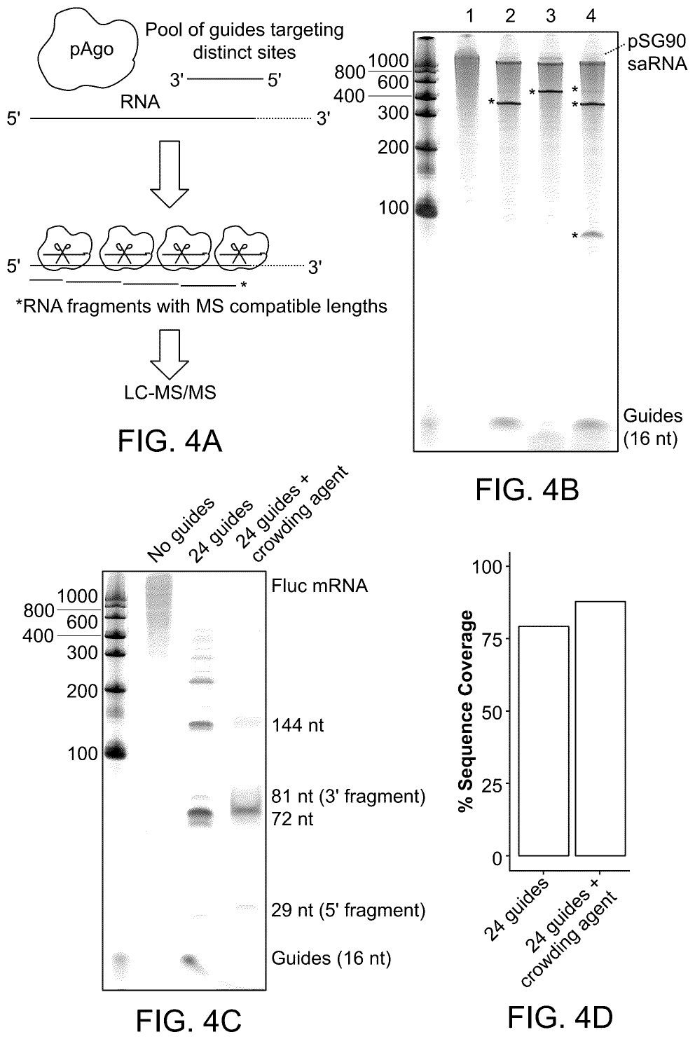

For the programmed cleavage of long RNAs into fragments of lengths between 70-110 nt (e.g., for analysis by full mass LC-MS/MS), guide sequences were multiplexed to site specifically cleave the substrate RNA simultaneously at the desired locations (schematic shown in FIG. 4A). Multiplexing of two guides (schematic shown in FIG. 4B) was tested in reactions containing 0.37 pmol of pSG90 saRNA in 1× ThermoPol reaction buffer with 1.85 pmol MpaAgo. Either no guide (FIG. 4C, lane 1), 4.63 pmol of guide 90.1 (FIG. 4C, lane 2), 4.63 pmol of guide 90.2 (FIG. 4C, lane 3), or an equimolar mix of 4.63 pmol (total 9.25 pmol) each of 90.1 and 90.2 (FIG. 4C, lane 4) where added and processed as described above.

In the presence of both guides, a new and predicted RNA fragment was successfully generated (shown as black asterisk in FIG. 4B, lane 4), while the cleavage product generated by guide 90.2 alone (shown as light grey asterisk in FIG. 4B) was efficiently cut at the expected location. These data suggest that multiplexing of guides to simultaneously cleave long RNA to desired lengths is possible, highly efficient and specific.

Agos programmed with more than two guides were multiplexed and shown to fully and simultaneously cleave a mRNA sequence into RNA fragments with appropriate sizes for LC-MS/MS analysis using 24 different guides targeting pre-determined sequences 72 nt apart in the FLuc mRNA incubated with MpaAgo (FIG. 4C). 0.37 pmol of FLuc mRNA were incubated with 3.7 pmol MpaAgo and 3.7 pmol of 24 pooled guides F1.1 to F1.24 (0.15 pmol of each guide) in 1× Thermopol reaction buffer without (FIG. 4C, lane 2) or with (FIG. 4C, lane 3) crowding agents for 30 minutes at 50° C. In the presence of the crowding agents, 100% of the FLuc mRNA was cleaved, resulting in the programmed fragment sizes (29 nt 5′ fragment, 81 nt 3′ fragment, 72 nt all other fragments). In addition, a faint band running at 144 nt indicates that not all sites were completely cleaved under the specific non-optimized conditions tested. However, the length of most cleavage fragments conforms with the lengths programmed via the 24 guides and is thus compatible with downstream LC-MS/MS analysis. Subsequent LC-MS/MS analysis of the cleaved fragments revealed that between 80% (24 guides multiplexed without crowding agents) and 89% (24 guides multiplexed with crowding agents) of the FLuc sequence were covered (FIG. 4D).

### Example 2: Conditions for RNA Cleavage Reactions by Ago

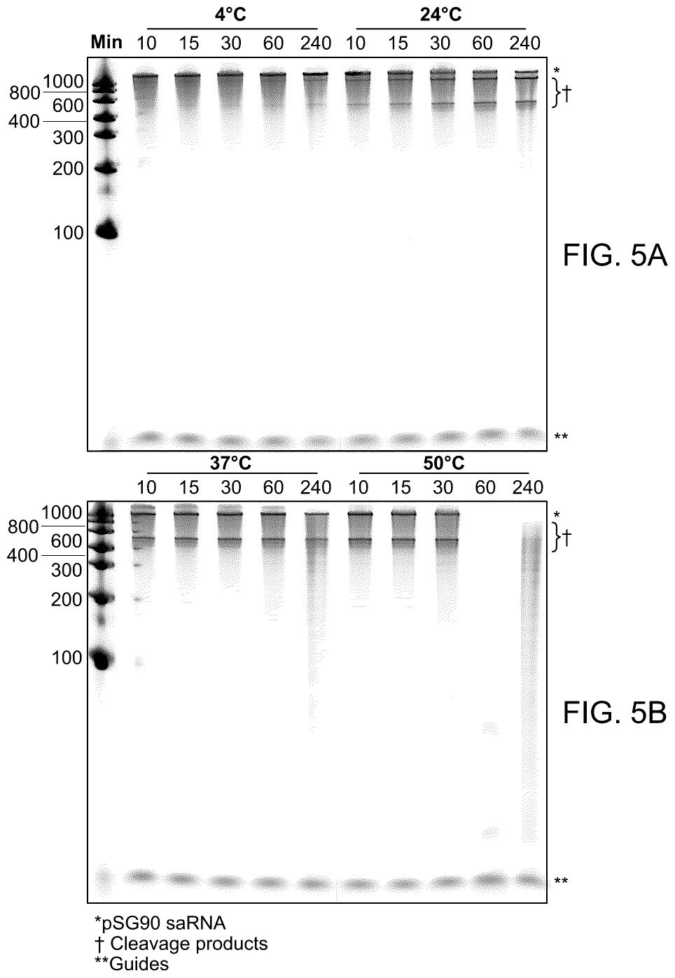

To assess the temperature and time ranges and requirements for the programmable Ago cleavage conditions, reactions containing 0.37 pmol of pSG90 saRNA, 1.85 pmol MpaAgo and 4.63 pmol guide 90.9 (Ago:guide ratio of 1:2.5) were prepared at 4° C., moved to a heat block, and incubated for 10 to 240 min at 4° C., 24° C., 37° C., and 50° C. as indicated above the gel (FIG. 5). At 4° C. incubation temperature, weak cleavage was only detected after 240 min. At 24° C., cleavage was observed after 10 min. However, even after 240 min of incubation at 24° C., approximately half of the substrate remained uncleaved. Cleavage efficiency was greatly improved at 37° C. incubation temperature, with the majority of substrate being cleaved after 10 min. The substrate was fully cleaved after 10 min incubation at 50° C. As expected, a 50° C. reaction temperature led to the Ago/guide independent, Mg²⁺ facilitated fragmentation of the substrate RNA when incubated for longer than 30 min.

### Example 3: Guide Removal from RNA Cleavage Reactions

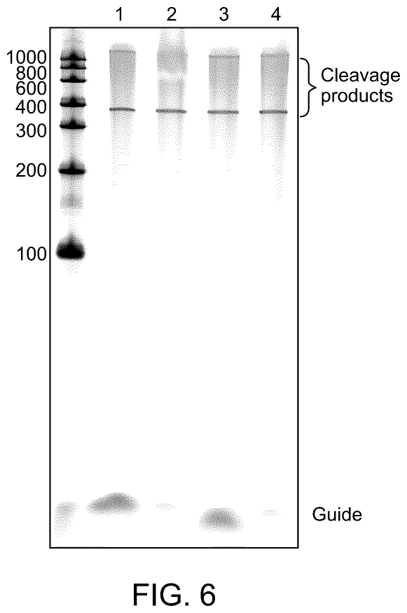

Certain applications may benefit from or require the removal of the DNA guides from the Ago reactions for downstream processing. In a single 50 μl reaction, 1.85 pmol of the pSG90 saRNA substrate were reacted with 9.25 pmol of MpaAgo and 46.25 pmol guide 90.1 in ThermoPol reaction buffer at 50° C. After 15 min, the reaction was split into four 10 μl aliquots and supplemented with various DNases to digest the DNA guide. No DNase was added to the reaction shown in FIG. 6, lane 1. 20 U of E. coli Exonuclease I (NEB #M0293S) was added to the reaction in lane 2, 10 U of Lambda Exonuclease (NEB #M0262S) was added to the reaction shown in lane 3, and 10 U E. coli Exonuclease I plus 2.5 U of Lambda Exonuclease were added to the reaction in lane 4. While Lambda Exonuclease did not remove the DNA guide, E. coli Exonuclease I either alone or in combination with Lambda Exonuclease led to an almost complete removal of guide. Residual guide in reactions containing E. coli Exonuclease I may represent Ago-bound guides that are not susceptible for exonucleases.

### Example 4: Precise 5′ Cleavage of mRNA by Programmable Agos for Cap Analysis

Analyzing and documenting the efficiency of mRNA 5′ capping is an essential quality control step in modern mRNA therapeutics manufacturing. Current methods of cap analysis usually combine UHPLC-MS/MS and fragmentation of the respective mRNA by methods including selective cleavage of the 5′ end by ribonuclease H in combination with a DNA probe, sequence specific RNases (e.g., RNase 4), deoxyribozymes, and ribozymes. This disclosure proposes the use of guided Agos to specifically and precisely cleave the cap-containing 5′ end of the substrate mRNA. This will result in a population of short RNA fragments which then can be size selected and further be analyzed via LC/MS-MS to determine capping efficiency.

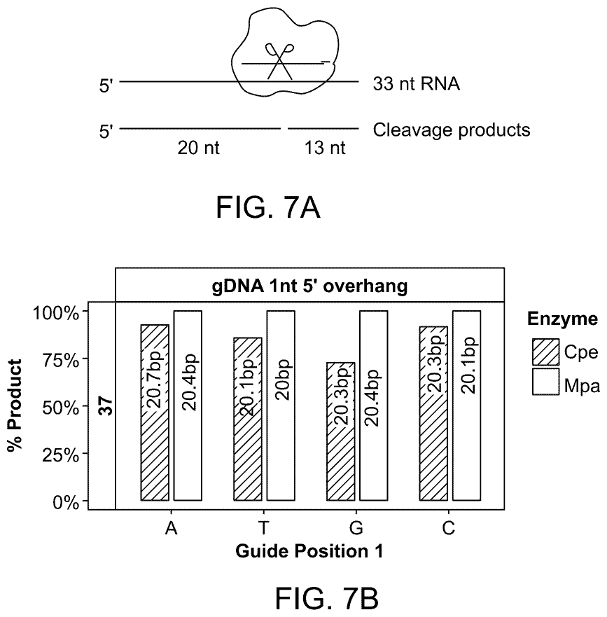

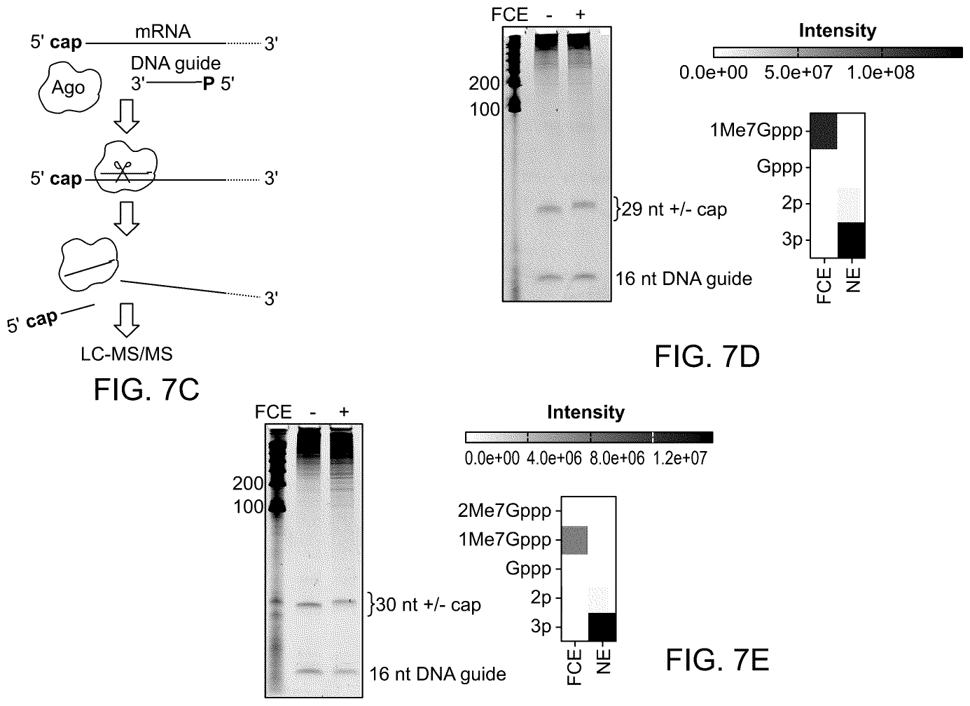

0.2 pmol of a short RNA representing 33 nt of the 5′-end of the FLuc mRNA (SEQ ID 29), 0.2 pmol of Mpa or CpeAgo were reacted in separate reactions with 1 pmol guides 8.1L15-A/C/T/G (SEQ ID NOS: 30-33) in Thermopol reaction buffer for 30 min at 37° C. The reactions were stopped by proteinase K treatment as described above and cleavage products analyzed by capillary electrophoresis. Guides were designed to cleave 20 nt downstream of the 5′ end and a single unpaired 5′ nucleotide (schematic shown in FIG. 7A). MpaAgo completely cleaved the substrate RNA at the expected position independent on whether the guide was fully complementary or not, while CpeAgo showed a slight reduction in cleavage efficiency when paired with the guide starting with GTP (SEQ ID 32; FIG. 7B). Both Agos specifically cleaved the substrate at the programmed/expected location, generating a 20 nt fragment that could be analyzed by LC-MS/MS.

This was also shown to be applicable to full length mRNA (workflow shown in FIG. 7C). Briefly, 1.66 pmol of FLuc mRNA (FIG. 7D) or pSG90 mRNA (FIG. 7E) were either treated or not treated with Faustovirus Capping Enzyme (FCE; NEB #M2081S) to install a 1Me7Gppp cap. 1.66 pmol of column purified capped and uncapped mRNA was then reacted with 16.6 pmol MpaAgo and 16.6 pmol guides (SEQ ID NO:13 for FLuc, SEQ-ID NO: 54 for pSG90) designed to cleave 29 nt or 30 nt, respectively, downstream of the 5′ end of the respective mRNA. After phosphorylation of the guides using T4 PNK (NEB #M0201S), the mRNA species were separately incubated at 50° C. in 1× ThermoPol buffer, glycerol, Murine RNase inhibitor (NEB #M0314S) with MpaAgo and the respective guides. After 30 min, the reactions were stopped by the addition of 0.08 U of Proteinase K (NEB #P8107S), followed by an incubation of 10 min at room temperature. After Ago induced cleavage of the 5′ ends, the cleavage fragments were either analyzed by denaturing gel electrophoresis or LC-MS/MS. Successful capping of the FLuc mRNA was detectable via gel electrophoresis on 15% TBE-urea gels (ThermoFisher #EC6885), with the capped fragment in the “FCE+” lane running slower in the gel (FIG. 7D, left panel). LC-MS/MS analysis revealed that the main 5′ structure in the FCE treated sample was 1Me7Gppp, while the uncapped FLuc mRNA 5′ end consisted of a triphosphate (FIG. 7D, right panel). Gel and LC-MS/MS analysis were also performed for the capped and uncapped pSG90 mRNA with similar results, showing that capped 5′ fragments can be detected via gel electrophoresis due to their slower migration in the gel, and further be analyzed by LC-MS/MS (FIG. 7E). Consistent with the LC-MS/MS data for FLuc, the 5′ end of uncapped pSG90 mRNA contained a triphosphate. While the majority of FCE capped pSG90 mRNA had a 1Me7Gppp cap, other cap species were also detected (e.g., 2Me7Gppp). These data indicate that pAgos can successfully be used to characterize 5′ capping efficiency, including detection and quantification of the cap analogs and reaction intermediates.

This method may be adapted to other applications, e.g., by facilitating the release of a 5′ RNA fragment containing a radioactive or fluorescent label, and/or the release of an RNA that is covalently bound/immobilized/tethered to a surface.

### Example 5: Use of Programmed Agos to Generate mRNA Populations with Homogenous 3′-Ends

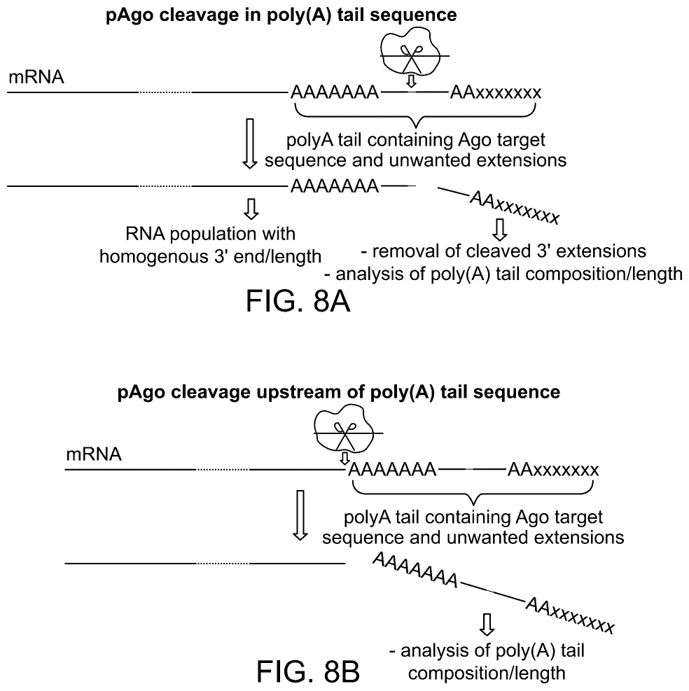

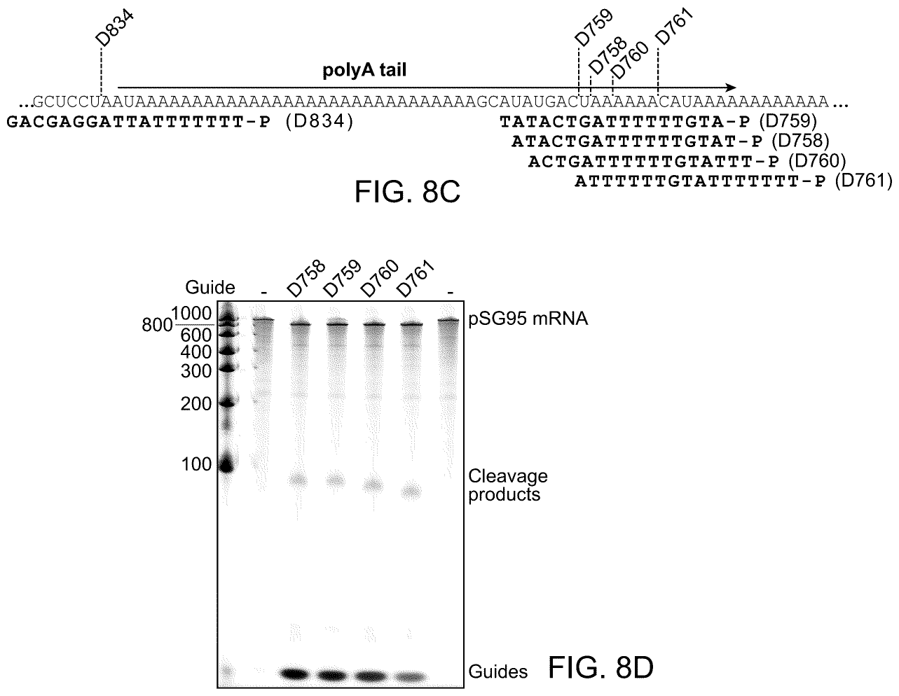

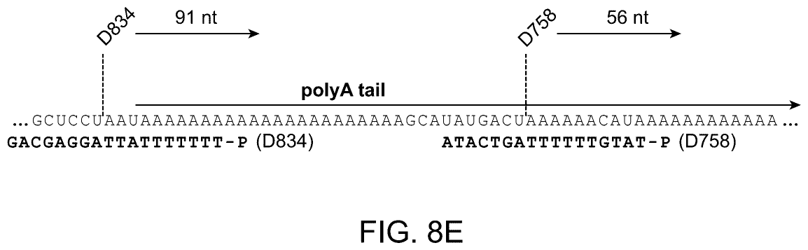

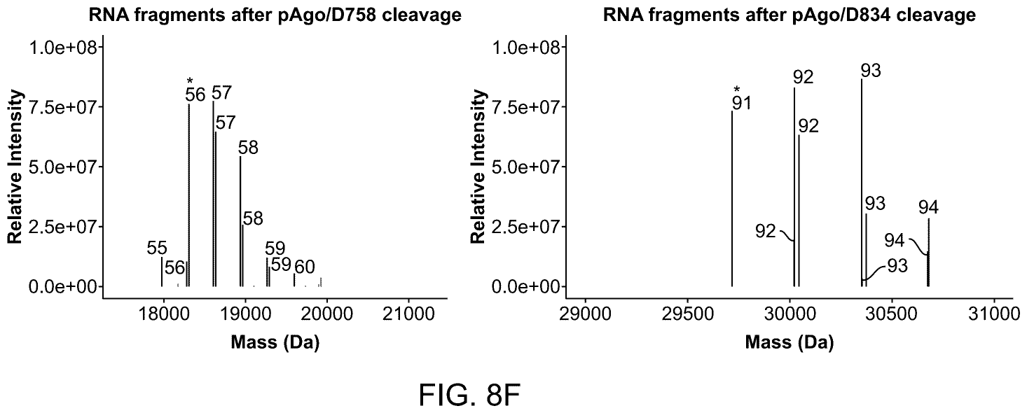

Undesired 3′ extensions by RNA polymerase that can have detrimental effects on the efficacy of therapeutic mRNAs are a common byproduct of the in vitro mRNA synthesis. Programming an Ago to cleave at a specific location in the poly(A) tail may be used to generate mRNA populations with homogenous poly(A) tail length and thus homogenous 3′-ends. Cleaved RNA extensions may be removed by various methods including, without limitation, HPLC purification, membrane filtration, size exclusion purification, precipitation, anion-exchange purification, gel electrophoresis, removal by a 5′-3′ exonuclease. Alternatively, a pAgo may be programmed to cleave immediately upstream of the poly(A) tail and the cleaved 3′ fragments may be analyzed by LC-MS/MS, for example, to determine the fragment sequence and length and/or to identify non-templated or templated 3′ additions (FIG. 8A).

FIG. 8B shows the sequence of a portion of the 3′ end, including nucleotides 602to 671, of a model mRNA (SEQ ID 23). Guides were designed that program pAgo to cleave the substrate RNA at positions 650, 649, 652, 656 of the respective target sequences, SEQ ID NOS: 48-51. 0.81 pmol of pSG95 epo mRNA were incubated with 4 pmol of MpaAgo, and 20 pmol of the respective guide in ThermoPol reaction buffer for 30 min at 50° C., treated with proteinase K as described above, and analyzed on a 6% Novex™ TBE-Urea gel (FIG. 8C). All guides resulted in highly efficient cleavage of the substrate at the expected positions, showing that even guides with low sequence diversity may be used to specifically and efficiently guide Agos to the respective/programmed cleavage site.

LC-MS/MS experiments were performed to analyze the poly(A) tail length and to identify non-templated or templated additions by the RNA polymerase during the in vitro transcription assay. For this, a substrate with a shortened poly(A) tail was generated that allows for the LC-MS/MS detection of additions to the poly(A) tail (pSG120; SEQ ID 60; FIG. 8D). Reactions were scaled up (67 pmol pSG120, 162 pmol MpaAgo, 800 pmol guide) and reacted in 400 μL 1× ThermoPol reaction buffer for 30 min at 50° C. Argonaute loaded with guide D834 (SEQ ID NO: 59) was used to cleave upstream of the poly(A) tail, completely removing the poly(A) tail and calculated to result in a 91 nt long 3′ cleavage fragment. Argonaute (pAgo) loaded with guide D758 (SEQ ID NO: 48) was used to cleave a sequence embedded in the poly(A) tail, calculated to result in a 56 nt long 3′ cleavage fragment. Reactions were stopped by addition of 0.8 units of proteinase K and incubation for 10 min at room temperature. Samples were column purified (NEB Monarch #T2040) and submitted for LC-MS/MS analysis. Fragments were separated and identified by LC-MS/MS. Encoded poly(A) tails were analyzed for non-templated extensions, which analysis revealed non-templated additions of one to four nucleotides (FIG. 8E).

In addition to the function disclosed above, trimming of undesired 3′-ends may be used to generate homogenous populations of RNA molecules that may be used as precise standards in e.g., electrophoretic, photometric, or mass spectrometric analysis. This approach may also be used to remove poly(A) tails from cellular mRNA by programming a pAgo with an oligo dT containing guide. This may lead to random trimming, and thus removal of the poly(A) tails of cellular mRNAs.

### Example 6: Release of Various Unique RNA Molecules or Multiple Copies of the Same RNA Molecule from a Single, Polycistronic in Vitro Transcribed RNA Molecule

Polycistronic in vitro transcribed RNA molecules for this Example 6 are defined by an in vitro transcribed single stranded RNA containing either multiple (>1) copies of the same RNA sequence and/or multiple (>1) different RNA sequences that are each separated by either the same or different RNA sequences representing the recognition site for either a single or different guide sequences. FIG. 9A illustrates an example in which multiple copies of the same RNA sequence that are separated by either a single or multiple unique recognition sequences for either a single or multiple unique guide sequences are encoded in a polycistronic RNA. Upon reacting the polycistronic RNA with an Ago programmed by either a single or multiple unique guide sequences, the copies of the RNA sequence are being cut apart and released as single molecules. In FIG. 9B the copies of a single RNA sequence have been replaced by different RNA sequences that upon cleavage with an Ago loaded with the respective guide/guides, releases the different RNA as separate molecules.

A schematic representation of an in vitro transcription template encoding a 1576 nt transcript (pSG106 RNA; SEQ ID NO:52) that contains five D770 guide (SEQ ID NO: 53) target sequences is shown in FIG. 9C. Successful cleavage of transcription products of this template by MpaAgo loaded with guide D770 would be expected to result in 6 RNA fragments with various lengths from 56 to 514 nt. 0.37 pmol of the pSG106 RNA were incubated with 3.7 pmol of MpaAgo and 18.7 pmol of guide 770 in 1× Thermopol reaction buffer without (FIG. 9D, lane 2) or with crowding agent (FIG. 9D, lane 3) and Murine RNase inhibitor for 30 min at 50° C. The control reaction in lane 1 did not contain the guide. Reactions were stopped by the addition of proteinase K and incubation at 37° C. for 10 min. After the addition of RNA loading dye and heat denaturation (5 min at 70° C.), the fragments were resolved by denaturing gel electrophoresis. The reaction containing Ago, guide, and the crowding agent resulted in the complete fragmentation of the pSG106 RNA into fragments equating the lengths programmed by guide 770. These data suggest that the combination of a single Ago with a single guide that targets multiple sites in one substrate results in the desired and programmed fragmentation of the substrate.

### Example 7: Enrichment of Specific RNAs from an RNA Pool

Site specific targeting of RNAs by guided Agos may also be used to enrich for RNAs of interest from a heterogeneous RNA pool followed by direct RNA sequencing using next generation sequencing by ONT Nanopore or Illumina based methods.

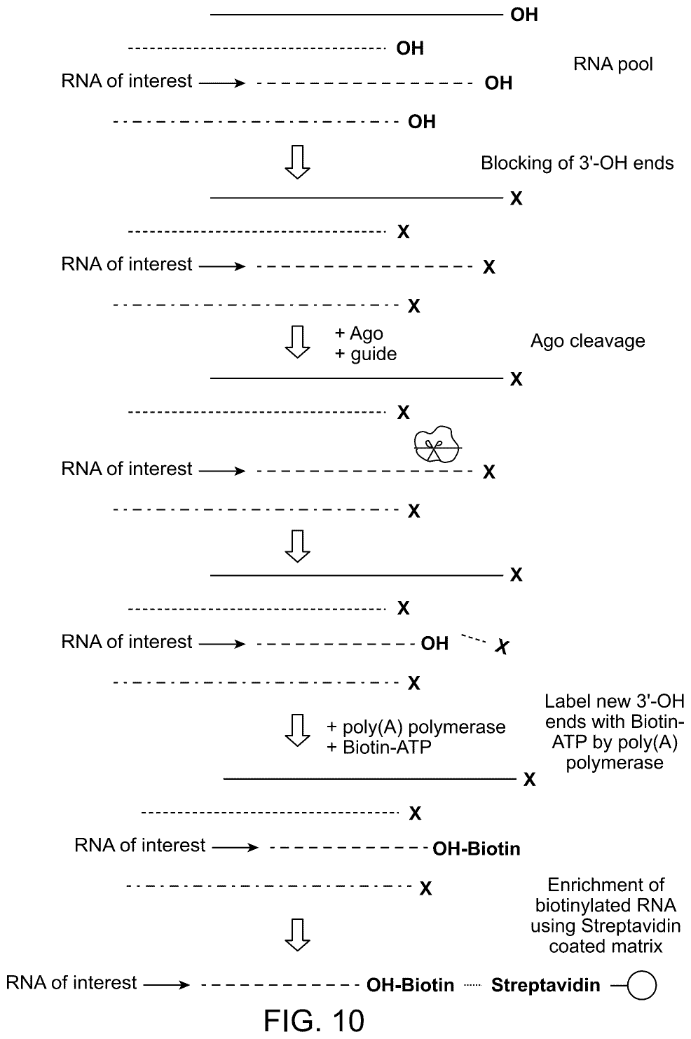

To enrich specific RNA species in a mixture of heterogenous RNA molecules, the 3′-OH ends of all RNA species in the mixture may be enzymatically modified or extended to alter the 3′ end to remove the 3′-OH (FIG. 10). This may be done, for example, by non-templated extension of the RNA 3′-OH with dNTPs using a polymerase. Subsequently, an Ago may be programmed via guide selection to cleave adjacent to the 3′ end of the RNA of interest, generating cleavage product with 3′-OH ends. Only the new 3′-OH ends can further be extended/biotinylated with Biotin-ATP by poly(A) polymerase since poly(A) polymerase requires a 3′-OH end for its polymerization activity. This will selectively biotinylate the RNA molecules of interest, which can subsequently be enriched for by affinity binding to a Streptavidin-coated matrix. After elution of the RNA of interest from the Streptavidin matrix, the RNA may be sequenced using the ONT Nanopore direct RNA sequencing protocol or Illumina sequencing using an oligo(dT) primer in a standard RNA library preparation. All other unwanted/uncleaved RNA species will not be biotinylated and thus not be sequenced.

A variation of this application omits the Biotin enrichment step. Since the ONT nanopore adapter requires a free 3′-OH to be ligated to RNA, RNAs with blocked 3′ ends cannot be ligated and thus will not be sequenced. Only RNAs with pAgo generated new 3′-OH ends will successfully ligate and be sequenced by ONT nanopore.

### Example 8: Specific Removal of RNA/DNA Adapter Dimers from Short RNA Libraries for Illumina Sequencing

The success Illumina sequencing of RNA may benefit from or even depend on adapter dimers being absent from the RNA to be sequenced. For example, if present, short adapter dimers may amplify rapidly in a subsequent PCR reaction and significantly reduce the amount of useful sequence information produced in any downstream sequencing reaction. Accordingly, it may be desirable to avoid adapter dimer formation and/or provide full removal of adapter dimers that may arise during RNA library prep. This may be particularly important when sequencing samples comprising low amounts of RNA and/or comprising valuable RNA. Specific pAgo cleavage at the junction between both ligated adapters will result in two fragments. These cleaved adapter dimers cannot be amplified in a PCR reaction, as each fragment is missing one PCR primer binding site, and thus will not be present in the final sequencing library. Guide selection to a region of the substrate that is uniquely formed by sequences of both individual adapters ensures that only adapter dimers and not correctly adapter-ligated RNA sample will be depleted.

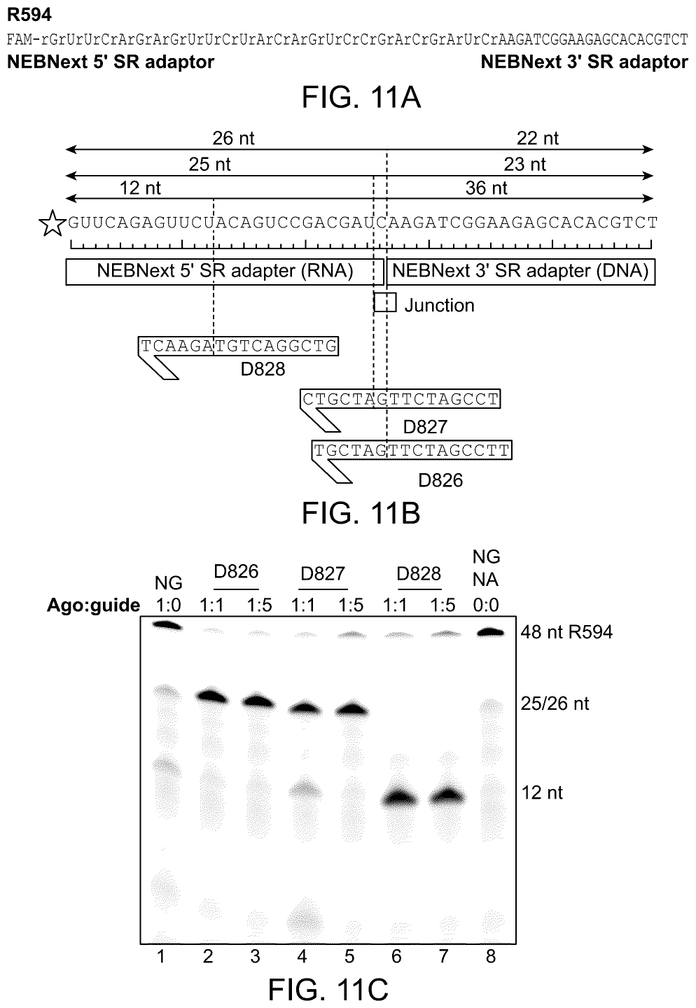

3.13 pmol of R594 substrate (SEQ ID NO: 55; FIG. 11A) were contacted with 15.7 pmol (FIG. 11C, lanes 1-7) or no MpaAgo (lane 8), that was programmed by binding to either 15.7 pmol (1:1 ratio Ago:guide) or 78.5 pmol (1:5 ratio Ago:guide) guide as indicated above the gel. The reactions were incubated for 15 min 50° C. in 1× ThermoPol reaction buffer, stopped by the addition of 0.4 units of Proteinase K and incubation at 24° C. for 10 min. After addition of RNA loading dye and heat denaturation for 4 min at 70° C., the samples were separated on a denaturing 15% TBU gel. The data show highly efficient cleavage of the exemplary RNA/DNA adapter dimer, no matter if the guide target sequence was located in the RNA only part of the substrate (D828) or at the junction, binding to both, the DNA and RNA part of the substrate (D826 and D827), suggesting that guide loaded MpaAgo specifically cleaves an RNA substrate even if the 5′ end of guide partially has to bind to a DNA sequence.

These data demonstrate that pAgos programmed with guides that target the junction of RNA/DNA adapter dimers may be used effectively to cleave unwanted adapter dimers and thus remove them from RNA library preparations.

1. A method for cleaving a single-stranded target RNA, the method comprising: contacting:
a single-stranded target RNA comprising a guide-recognition sequence;
an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and
a guide having a sequence complementary to the guide-recognition sequence, wherein the guide is operatively bound to the Argonaute forming an Argonaute:guide complex,
to produce cleavage products.

2. A method according to claim 1, wherein cleavage products include fragments of the single-stranded RNA.

3. A method according to claim 1, wherein the single-stranded RNA comprises at least 5000 nucleotides, wherein the single-stranded RNA has one guide recognition sequence.

4. A method according to claim 1, wherein the single-stranded RNA further comprises one or more modified nucleotides.

5. A method according to claim 1, wherein the guide has a sequence selected from any of SEQ ID NOS: 2-11, 13-22, 24-28, 30-36, 38-42, and 48-51.

6. A method according to claim 1, wherein the single-stranded RNA comprises a messenger RNA (mRNA), a ribosomal RNA (rRNA), a transfer RNA (tRNA), a small RNA (sRNA), a microRNA (miRNA), a long noncoding RNA (IncRNA), a circular RNA (circRNA), a transfer RNA (tRNA), an aptamer RNA, an antisense RNA, a silencing RNA (siRNA), a guide RNA (gRNA), or a therapeutic RNA.

7. A method according to claim 1, wherein the single-stranded RNA is a polycistronic template comprising at least five guide recognition sequences that each corresponds to the Argonaute: guide complex.

8. A method according to claim 1, wherein at least one of the cleavage products comprises an IVT template.

9. A method according to claim 1, further comprising analyzing at least one cleavage product.

10. A method according to claim 9, wherein analyzing at least one cleavage product further comprises analyzing the at least one cleavage product by electrophoretic, photometric, and/or mass spectrometric analysis.

11. A method according to claim 1, wherein the contacting excludes contacting a single-stranded binding protein with any of the single-stranded target RNA, the Argonaute, or the guide.

12. A composition comprising:
a single-stranded target RNA comprising a guide-recognition sequence;
an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and
a guide having a sequence complementary to a single-stranded target RNA guide-recognition sequence, wherein the guide is operatively bound to the Argonaute forming an Argonaute: guide complex.

13. A composition according to claim 12 further comprising a reaction buffer.

14. A composition according to claim 12, further comprising a buffering agent selected from HEPES, MES, MOPS, TAPS, tricine, Tris, ACES, ADA, BES, Bicine, CAPS, CHES, DIPSO, EPPS, MOPSO, PIPES, POPSO, TAPS, and TAPSO.

15. A composition according to claim 12, wherein the composition does not comprise a whole cell or a cell extract.

16. A composition according to claim 12, wherein the composition is free of any single-stranded binding protein.

17. A composition according to claim 12, wherein composition has a form selected from a dried form, a freeze dried form, a lyophilized form, a crystalline form, an aqueous form, and an immobilized form.

18. A kit comprising:
an Argonaute selected from the group consisting of an Aquifex aeolicus Argonaute, a Bacteroidetes bacterium Argonaute (BbAgo), a Chitinophaga costaii Argonaute (CcAgo), a Chitinophagaceae bacterium Argonaute (ChbAgo), a Chlostridium perfringens Argonaute (CpeAgo), a Mucilaginibacter paludis Argonaute (MpaAgo), and a Thermus thermophilus Argonaute; and
a guide having a sequence complementary to at least a portion of a single-stranded target RNA, wherein the guide has a sequence selected from any of SEQ ID NOS: 2-11,13-22, 24-28, 30-36, 38-42 and 48.51.

19. A kit according to claim 18, further comprising a buffering agent selected from HEPES, MES, MOPS, TAPS, tricine, Tris, ACES, ADA, BES, Bicine, CAPS, CHES, DIPSO, EPPS, MOPSO, PIPES, POPSO, TAPS, and TAPSO.

20. A kit according to claim 18, wherein the Argonaute and/or the guide has a form selected from a dried form, a freeze dried form, a lyophilized form, a crystalline form, an aqueous form, and an immobilized form.


SEQUENCE LISTING (60 sequences)

SEQ ID NO: 1 - RNA, 1678 nt, synthetic construct
  source 1..1678: mol_type = other RNA
ggtgacggcgtagtacacactattgaatcaaacagccgaccaattgcactaccatcacaatggagaagccagtagtaaacgtagacgtagacccccagagtccgtttgtcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgagcaccagtatcattgtgtctgccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggcttcgacaccacccagttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgagggcttcttgctatgcaaagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaattgtcattaacggtaggactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgctgcaggtctcggaggaattagtcatggaggccattttccgaatcggattttgtttttaatatttcaaaaaaaaaaaaaaaaaaaaaaaaaaaaaagcatatgactaaaaaaaaaaaaaaaaaaaaaaaaaaaaaaaaaaaaaaaaaaaaaaaaaaaaaaaaaaaaaaaaaaaaaa

SEQ ID NO: 2 - DNA, 16 bp, synthetic construct
  source 1..16: mol_type = other DNA
ctttttccgccagttt

SEQ ID NO: 3 - DNA, 16 bp, Synthetic construct
  source 1..16: mol_type = other DNA
ttgtgaaagcagagcg

SEQ ID NO: 4 - DNA, 16 bp, synthetic construct
  source 1..16: mol_type = other DNA
ccgcacgcctttcata

SEQ ID NO: 5 - DNA, 16 bp, synthetic construct
  source 1..16: mol_type = other DNA
ggactttctcgtcggc

SEQ ID NO: 6 - DNA, 16 bp, synthetic construct
  source 1..16: mol_type = other DNA
cgcgacccgggcttca

SEQ ID NO: 7 - DNA, 16 bp, synthetic construct
  source 1..16: mol_type = other DNA
cgactgctttccattc

SEQ ID NO: 8 - DNA, 16 bp, synthetic construct
  source 1..16: mol_type = other DNA
ccgcgtatcccacggt

SEQ ID NO: 9 - DNA, 16 bp, synthetic construct
  source 1..16: mol_type = other DNA
atggtggccgggatgt

SEQ ID NO: 10 - DNA, 16 bp, synthetic construct
  source 1..16: mol_type = other DNA
cctaccgttaatgaca

SEQ ID NO: 11 - DNA, 16 bp, synthetic construct
  source 1..16: mol_type = other DNA
cgttatcaagatcatc

SEQ ID NO: 12 - RNA, 1766 nt, synthetic construct
  source 1..1766: mol_type = other RNA
gggtctagaaataattttgtttaactttaagaaggagatataaccatgaaaatcgaagaaggtaaaggtcaccatcaccatcaccacggatccatggaagacgccaaaaacataaagaaaggcccggcgccattctatcctctagaggatggaaccgctggagagcaactgcataaggctatgaagagatacgccctggttcctggaacaattgcttttacagatgcacatatcgaggtgaacatcacgtacgcggaatacttcgaaatgtccgttcggttggcagaagctatgaaacgatatgggctgaatacaaatcacagaatcgtcgtatgcagtgaaaactctcttcaattctttatgccggtgttgggcgcgttatttatcggagttgcagttgcgcccgcgaacgacatttataatgaacgtgaattgctcaacagtatgaacatttcgcagcctaccgtagtgtttgtttccaaaaaggggttgcaaaaaattttgaacgtgcaaaaaaaattaccaataatccagaaaattattatcatggattctaaaacggattaccagggatttcagtcgatgtacacgttcgtcacatctcatctacctcccggttttaatgaatacgattttgtaccagagtcctttgatcgtgacaaaacaattgcactgataatgaattcctctggatctactgggttacctaagggtgtggcccttccgcatagaactgcctgcgtcagattctcgcatgccagagatcctatttttggcaatcaaatcattccggatactgcgattttaagtgttgttccattccatcacggttttggaatgtttactacactcggatatttgatatgtggatttcgagtcgtcttaatgtatagatttgaagaagagctgtttttacgatcccttcaggattacaaaattcaaagtgcgttgctagtaccaaccctattttcattcttcgccaaaagcactctgattgacaaatacgatttatctaatttacacgaaattgcttctgggggcgcacctctttcgaaagaagtcggggaagcggttgcaaaacgcttccatcttccagggatacgacaaggatatgggctcactgagactacatcagctattctgattacacccgagggggatgataaaccgggcgcggtcggtaaagttgttccattttttgaagcgaaggttgtggatctggataccgggaaaacgctgggcgttaatcagagaggcgaattatgtgtcagaggacctatgattatgtccggttatgtaaacaatccggaagcgaccaacgccttgattgacaaggatggatggctacattctggagacatagcttactgggacgaagacgaacacttcttcatagttgaccgcttgaagtctttaattaaatacaaaggatatcaggtggcccccgctgaattggaatcgatattgttacaacaccccaacatcttcgacgcgggcgtggcaggtcttcccgacgatgacgccggtgaacttcccgccgccgttgttgttttggagcacggaaagacgatgacggaaaaagagatcgtggattacgtcgccagtcaagtaacaaccgcgaaaaagttgcgcggaggagttgtgtttgtggacgaagtaccgaaaggtcttaccggaaaactcgacgcaagaaaaatcagagagatcctcataaaggccaagaagggcggaaagtccaaactcgagtaaggttaacctgcaggagg

SEQ ID NO: 13 - DNA, 16 bp, synthetic construct
  source 1..16: mol_type = other DNA
atctccttcttaaagt

SEQ ID NO: 14 - DNA, 16 bp, synthetic construct
  source 1..16: mol_type = other DNA
catagccttatgcagt

SEQ ID NO: 15 - DNA, 16 bp, synthetic construct
  source 1..16: mol_type = other DNA
gattctgtgatttgta

SEQ ID NO: 16 - DNA, 16 bp, synthetic construct
  source 1..16: mol_type = other DNA
cactacggtaggctgc

SEQ ID NO: 17 - DNA, 16 bp, synthetic construct
  source 1..16: mol_type = other DNA
gggaggtagatgagat

SEQ ID NO: 18 - DNA, 16 bp, synthetic construct
  source 1..16: mol_type = other DNA
ggcatgcgagaatctg

SEQ ID NO: 19 - DNA, 16 bp, synthetic construct
  source 1..16: mol_type = other DNA
ttcttcaaatctatac

SEQ ID NO: 20 - DNA, 16 bp, synthetic construct
  source 1..16: mol_type = other DNA
aggtgcgcccccagaa

SEQ ID NO: 21 - DNA, 16 bp, synthetic construct
  source 1..16: mol_type = other DNA
aacaactttaccgacc

SEQ ID NO: 22 - DNA, 16 bp, synthetic construct
  source 1..16: mol_type = other DNA
cttgtcaatcaaggcg

SEQ ID NO: 23 - DNA, 16 bp, synthetic construct
  source 1..16: mol_type = other DNA
ggggtgttgtaacaat

SEQ ID NO: 24 - DNA, 16 bp, synthetic construct
  source 1..16: mol_type = other DNA
tttcgcggttgttact

SEQ ID NO: 25 - DNA, 16 bp, synthetic construct
  source 1..16: mol_type = other DNA
gtttttggcgtcttcc

SEQ ID NO: 26 - DNA, 16 bp, synthetic construct
  source 1..16: mol_type = other DNA
cgcgtacgtgatgttc

SEQ ID NO: 27 - DNA, 16 bp, synthetic construct
  source 1..16: mol_type = other DNA
aactgcaactccgata

SEQ ID NO: 28 - DNA, 16 bp, synthetic construct
  source 1..16: mol_type = other DNA
aataattttctggatt

SEQ ID NO: 29 - DNA, 16 bp, synthetic construct
  source 1..16: mol_type = other DNA
ggaattcattatcagt

SEQ ID NO: 30 - DNA, 16 bp, synthetic construct
  source 1..16: mol_type = other DNA
accgtgatggaatgga

SEQ ID NO: 31 - DNA, 16 bp, synthetic construct
  source 1..16: mol_type = other DNA
gaatgaaaatagggtt

SEQ ID NO: 32 - DNA, 16 bp, synthetic construct
  source 1..16: mol_type = other DNA
gagcccatatccttgt

SEQ ID NO: 33 - DNA, 16 bp, synthetic construct
  source 1..16: mol_type = other DNA
taattcgcctctctga

SEQ ID NO: 34 - DNA, 16 bp, synthetic construct
  source 1..16: mol_type = other DNA
caagcggtcaactatg

SEQ ID NO: 35 - DNA, 16 bp, synthetic construct
  source 1..16: mol_type = other DNA
aacaacaacggcggcg

SEQ ID NO: 36 - DNA, 16 bp, synthetic construct
  source 1..16: mol_type = other DNA
ttttcttgcgtcgagt

SEQ ID NO: 37 - RNA, 723 nt, synthetic construct
  source 1..723: mol_type = other RNA
gggcttgcttgttctttttgcagaagctcagaataaacgctcaactttggcaccgccgcacaaccggatgcgccgagcactgcagcctcaacgagaacatcaccgtaccggacacaaaggtcaactttacgcatggaagagaatggaagtaggacagcaggccgtcgaagtgtgcaggggctcgcgctgctgagcgaggcggtgctgcggggccaggccctcctcgtcaacagcagccagccgtgggagcccctccaactgcacgtcgacaaagcggtgagcgggctccgcagcctgacgacgctgctgcgggccctgggcgcacaaaaggaggccatcagcccgcccgacgcggccagcgcggcaccccccgaacgatcaccgcggacacgttcaggaagctgttagagtgtacagcaacttcctcgcggaaagctgaaactgtacaccggcgaagcgtgcaggacaggggaccgctaggactgactaggattggttaccactaaaccagcctcaagaacacccgaatggagtctctaagctacataataccaacttacacttacaacatgtgtcccccaaactgtagccattcgtatctgctcctaataaaaaaaaaaaaaaaaaaaaaaaaaaaaaagcatatgactaaaaaacataaaaaaaaaaaaaaaaaaaaaaaaaaaaaaaaaaaaaaaaaaaaaaaaaaaaaaaaaaaaagct

SEQ ID NO: 38 - DNA, 16 bp, synthetic construct
  source 1..16: mol_type = other DNA
gtaaagttgacctttg

SEQ ID NO: 39 - DNA, 16 bp, synthetic construct
  source 1..16: mol_type = other DNA
gctcccacggctggct

SEQ ID NO: 40 - DNA, 16 bp, synthetic construct
  source 1..16: mol_type = other DNA
ggggtgccgcgctggc

SEQ ID NO: 41 - DNA, 16 bp, synthetic construct
  source 1..16: mol_type = other DNA
ctagtcagtcctagcg

SEQ ID NO: 42 - DNA, 16 bp, synthetic construct
  source 1..16: mol_type = other DNA
attaggagcagatacg

SEQ ID NO: 43 - RNA, 33 nt, synthetic construct
  source 1..33: mol_type = other RNA
gggtctagaaataattttgtttaactttaagaa

SEQ ID NO: 44 - DNA, 16 bp, synthetic construct
  source 1..16: mol_type = other DNA
atcttaaagttaaaca

SEQ ID NO: 45 - DNA, 16 bp, synthetic construct
  source 1..16: mol_type = other DNA
ttcttaaagttaaaca

SEQ ID NO: 46 - DNA, 16 bp, synthetic construct
  source 1..16: mol_type = other DNA
gtcttaaagttaaaca

SEQ ID NO: 47 - DNA, 16 bp, synthetic construct
  source 1..16: mol_type = other DNA
ctcttaaagttaaaca

SEQ ID NO: 48 - DNA, 17 bp, synthetic construct
  source 1..17: mol_type = other DNA
tatgttttttagtcata

SEQ ID NO: 49 - DNA, 17 bp, synthetic construct
  source 1..17: mol_type = other DNA
atgttttttagtcatat

SEQ ID NO: 50 - DNA, 17 bp, synthetic construct
  source 1..17: mol_type = other DNA
tttatgttttttagtca

SEQ ID NO: 51 - DNA, 17 bp, synthetic construct
  source 1..17: mol_type = other DNA
tttttttatgtttttta

SEQ ID NO: 52 - RNA, 1576 nt, synthetic construct
  source 1..1576: mol_type = other RNA
ggtgacggcgtagtacacactattgaatcaaacagccgaccaattgcactaccatcacaatggagaagccagtagtaaacgtagacgtagacctgtcattaacggtaggcgtgcaactgcaaaaaagcttcccgcaatttgaggtagtagcacagcaggtcactccaaatgaccatgctaatgccagagcattttcgcatctggccagtaaactaatcgagctggaggttcctaccacagcgacgatcttggacataggcagcgcaccggctcgtagaatgttttccgaggacctgtcattaacggtaggccccatgcgtagtccagaagacccggaccgcatgatgaaatacgccagtaaactggcggaaaaagcgtgcaagattacaaacaagaacttgcatgagaagattaaggatctccggaccgtacttgatacgccggatgctgaaacaccatcgctctgctttcacaacgatgttacctgcaacatgcgtgccgaatattccgtcatgcaggacgtgtatatcaacgctcccggaactatctatcatcaggctatgaaaggcgtgcggaccctgtactggattggacctgtcattaacggtagggttcatgttctcggctatggcaggttcgtaccctgcgtacaacaccaactgggccgacgagaaagtccttgaagcgcgtaacatcggactttgcagcacaaagctgagtgaaggtaggacaggaaaattgtcgataatgaggaagaaggagttgaagcccgggtcgcgggtttatttctccgtaggatcgacactttatccagaacacagagccagcttgcagagctggcatcttccatcggtgttccacttgaatggaaagcagtcgtacacttgccgctgtgatacagtggtgagttgcgaaggctacgtagtgaagaaaatcaccatcagtcccgggatcacgggagaaaccgtgggatacgcggttacacacaatagcgaggacctgtcattaacggtaggagttactgacacagtaaaaggagaacgggtatcgttccctgtgtgcacgtacatcccggccaccatatgcgatcagatgactggtataatggccacggatatatcacctgacgatgcacaaaaacttctggttgggctcaaccagcgaatgcttcttgctatgcaaactaacaggaacaccaacaccatgcaaaattaccttctgccgatcatagcacaagggttcagcaaatgggctaaggagcgcaaggatgatcttgataacgagaaaatgctgggtactagagaacgcaagcttacgtatggctgcttgtgggcgtttcgcactaagaaagtacattcgttttatcgcccacctggaacgcagacctgcgtaaaagtcccagcctcttttagcgcttttcccatgtcgtccgtatggacgacctctttgcccatgtcgctgaggcagaaattgaaactggcattgcaaccaaagaaggaggaaaaactgcgacctgtcattaacggtaggattagtcatggaggccattttccgaatcggattttgtttttaatat

SEQ ID NO: 53 - DNA, 20 bp, synthetic construct
  source 1..20: mol_type = other DNA
nctaccgttaatgacaggtc

SEQ ID NO: 54 - DNA, 16 bp, synthetic construct
  source 1..16: mol_type = other DNA
gtcggctgtttgattc

SEQ ID NO: 55 - DNA, 48 bp, synthetic construct
  source 1..48: mol_type = other DNA
  misc_feature 1..27: note = RNA
  misc_feature 28..48: note = DNA
gttcagagttctacagtccgacgatcaagatcggaagagcacacgtct

SEQ ID NO: 56 - DNA, 16 bp, synthetic construct
  source 1..16: mol_type = other DNA
ttccgatcttgatcgt

SEQ ID NO: 57 - DNA, 16 bp, synthetic construct
  source 1..16: mol_type = other DNA
tccgatcttgatcgtc

SEQ ID NO: 58 - DNA, 16 bp, synthetic construct
  source 1..16: mol_type = other DNA
gtcggactgtagaact

SEQ ID NO: 59 - DNA, 18 bp, synthetic construct
  source 1..18: mol_type = other DNA
tttttttattaggagcag

SEQ ID NO: 60 - RNA, 698 nt, synthetic construct
  source 1..698: mol_type = other RNA
gggcttgcttgttctttttgcagaagctcagaataaacgctcaactttggcaccgccgcacaaccggatgcgccgagcactgcagcctcaacgagaacatcaccgtaccggacacaaaggtcaactttacgcatggaagagaatggaagtaggacagcaggccgtcgaagtgtgcaggggctcgcgctgctgagcgaggcggtgctgcggggccaggccctcctcgtcaacagcagccagccgtgggagcccctccaactgcacgtcgacaaagcggtgagcgggctccgcagcctgacgacgctgctgcgggccctgggcgcacaaaaggaggccatcagcccgcccgacgcggccagcgcggcaccccccgaacgatcaccgcggacacgttcaggaagctgttagagtgtacagcaacttcctcgcggaaagctgaaactgtacaccggcgaagcgtgcaggacaggggaccgctaggactgactaggattggttaccactaaaccagcctcaagaacacccgaatggagtctctaagctacataataccaacttacacttacaacatgtgtcccccaaactgtagccattcgtatctgctcctaataaaaaaaaaaaaaaaaaaaaaagcatatgactaaaaaacataaaaaaaaaaaaaaaaaaaaaaaaaaaaaaaaaaaagctatcggatc